(19) United States
(12) Patent Application Publication
(10) Pub. No.: US 2011/0165109 A1
(43) Pub. Date: Jul. 7, 2011

(54) AGENT FOR FIBERS CONTAINING KERATIN, COMPRISING AT LEAST ONE SPECIFIC AMPHIPHILIC CATIONIC POLYMER AND AT LEAST ONE ADDITIONAL FILM-FORMING AND/OR STABILIZING POLYMER SELECTED FROM CHITOSAN AND DERIVATES THEREOF

(76) Inventors: Burkhard Mueller, Hamburg (DE); Pamela Kaftan, Hamburg (DE)

(21) Appl. No.: 13/029,152

(22) Filed: Feb. 17, 2011

Related U.S. Application Data
(63) Continuation of application No. PCT/EP2009/059351, filed on Jul. 21, 2009.

(30) Foreign Application Priority Data
Aug. 18, 2008 (DE) 10 2008 038 107.1

(51) Int. Cl. A61K 8/81; A61Q 5/00

(52) U.S. Cl. 424/70.19

(57) ABSTRACT

Agent for treating fibers containing keratin, particularly human hair containing in a cosmetically acceptable carrier: (a) an amphiphilic, cationic polymer having a structural unit of formula (I), a structural unit of formula (II), a structural unit of formula (III) and a structural unit of formula (IV), wherein R¹ and R⁴ independently represent hydrogen or a methyl group, X¹ and X² independently represent oxygen or an NH group, A¹ and A² independently represent an ethane-1,2-diyl, propane-1,3-diyl or butane-1,4-diyl group, R², R³, R⁵ and R⁶ independently represent a (C₁ to C₄)-alkyl group, R⁷ represents a (C₈ to C₃₀)-alkyl group, and; (b) at least one film-forming and/or stabilizing polymer chosen from chitosan and derivatives thereof. The invention also relates to use of agents for temporarily styling hair and for haircare, particularly as an aerosol hairspray or aerosol hair mousse.

### CROSS REFERENCE TO RELATED APPLICATIONS

The present application is a continuation of International Patent Application No. PCT/EP2009/059351 filed 21 Jul. 2009, which is claims priority to German Patent Application No. 10 2008 038 107.1 filed 18 Aug. 2008, both of which are incorporated herein by reference.

The present invention relates to agents for treating hair comprising a combination of at least one specific amphiphilic, cationic polymer with at least one film-forming cationic and/or setting polymer chosen from chitosan and its derivatives, use of these agents for temporary shaping and/or care of keratin-containing fibers, and aerosol hair sprays/foams based on these agents.

Keratin-containing fibers include all animal hair (e.g., wool, horsehair, angora hair, furs, feathers and products or fabrics produced from them). However, keratinic fibers preferably concern human hair.

Today, a suitably looking hairstyle is generally regarded as an essential part of a well groomed appearance. Based on actual fashion trends, time and again hairstyles are considered chic, which, for many types of hair, can only be formed or sustained over a longer period of up to several days by the use of certain consolidating materials. Thus, hair treatments, which provide a permanent or temporary hairstyling, play an important role. Temporary styling intended to provide a good hold, without compromising the healthy appearance of the hair, such as, for example the gloss, can be obtained for example by the use of hairsprays, hair waxes, hair gels, hair foams, setting lotions etc.

Suitable compositions for temporary hairstyling usually comprise synthetic polymers as the styling component. Preparations comprising a dissolved or dispersed polymer can be applied on the hair by means of propellants or by a pumping mechanism. Hair gels and hair waxes in particular are however not generally applied directly on the hair, but rather dispersed with a comb or by hand.

The most important property of an agent for the temporary styling of keratin fibers, in the following also called styling agents, consists in giving the treated fibers the strongest possible hold in the created shape. If the keratinic fibers concern human hair, then one also speaks of a strong hairstyle hold or a high degree of hold of the styling agent. The styling hold is determined essentially by the type and quantity of the synthetic polymer used, but there may also be an influence from the other components of the styling agent.

In addition to a high degree of hold, styling agents must fulfill a whole series of additional requirements. These requirements can be broadly subdivided into properties on the hair, properties of the formulation in question, e.g. properties of the foam, the gel or the sprayed aerosol, and properties that concern the handling of the styling agent, wherein particular importance is attached to the properties on the hair. In particular, moisture resistance, low stickiness and a balanced conditioning effect should be mentioned. Furthermore, a styling agent should be universally applicable for as many types of hair as possible.

To do justice to the various requirements, various synthetic polymers have already been developed and are being used in styling agents. These polymers can be subdivided into cationic, anionic, non-ionic and amphoteric film-forming and/or setting polymers. Ideally these polymers form a polymer film when applied to hair, imparting on the one hand a strong hold to the hairstyle but on the other hand also being sufficiently flexible not to break under stress. If the polymer film is too brittle, so-called film plaques develop, i.e. residues that are shed with movement of the hair and give the impression that the user of the respective styling agent has dandruff.

To develop styling agents that in combination have all the desired properties still presents problems. This is particularly true for the combination of a strong hold on the one hand and on the other hand a simple, uniform application onto the keratin-containing fibers.

Accordingly, the object of the present invention was to provide an agent for the temporary shaping and/or for the care of keratinic fibers which is characterized by a high degree of hold or by a high care action and in particular possesses an excellent handleability during the application onto the keratin-containing fibers.

It has now been surprisingly found that this can be achieved by a combination of specific polymers. In addition, the compositions obtained with this combination actually exhibit a transparency that is visible to the naked eye.

Accordingly, a first subject matter of the present invention is an agent for treating keratin-containing fibers, especially human hair, comprising in a cosmetically acceptable carrier
(a) at least one amphiphilic, cationic polymer having at least one structural unit of Formula (I), at least one structural unit of Formula (II), at least one structural unit of Formula (III), and at least one structural unit of Formula (IV),

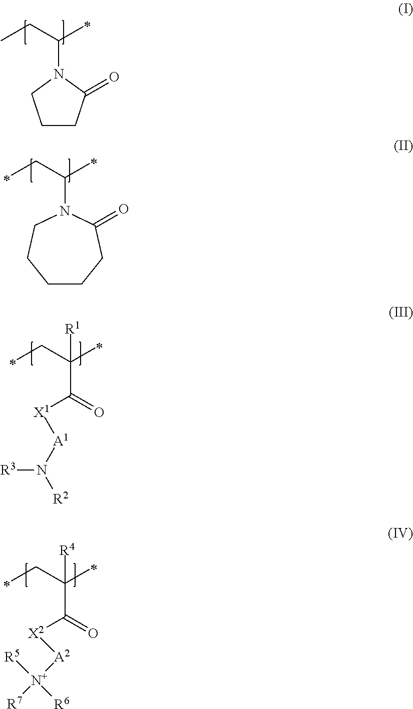

wherein
R¹ and R⁴ are, independently of one another, a hydrogen atom or a methyl group,
X¹ and X² are, independently of one another, an oxygen atom or an NH group,
A¹ and A² are, independently of one another, an ethane-1,2-diyl, propane-1,3-diyl or butane-1,4-diyl group,
R², R³, R⁵ and R⁶ are, independently of one another, a (C₁ to C₄) alkyl group, and
R⁷ is a (C₈ to C₃₀) alkyl group, and
(b) at least one film-forming and/or setting polymer chosen from chitosan and its derivatives.

Film-forming polymers refer to those polymers that, on drying, leave a continuous film on the skin, hair or nails. These types of film-former can be used in a wide variety of cosmetic products such as make up masks, make up, hair sets, hair sprays, hair gels, hair waxes, hair conditioners, shampoos or nail varnishes. Those polymers are particularly preferred, which are sufficiently soluble in alcohol or water/alcohol mixtures, so that they are present in completely dissolved form in the agents. The film-forming polymers can be of synthetic or of natural origin.

According to the invention, film-forming polymers further refer to those polymers that, when used in concentrations of 0.1 to 20 wt. % in aqueous, alcoholic or aqueous alcoholic solution, are able to separate out a transparent polymer film on the hair.

Setting polymers contribute to the hold and/or to the creation of the hair volume and the hair body of the whole hairstyle. These polymers are film-forming polymers at the same time and therefore generally typical substances for styling hair treatment compositions such as hair sets, hair foams, hair waxes, hair sprays. The film formation can be in completely selected areas and bond only some fibers together.

The curl-retention test is frequently used as a test method for the setting action.

In the above Formulae and all Formulae below, the symbol * signifies a chemical bond that stands for a free valence of the corresponding structural fragment.

According to the invention, the term “derivative” refers to derivatives of a chemical compound (e.g., chitosan) which can be synthesized from this chemical compound by the structural modification of a functional group of said chemical compound.

To compensate for the positive charge on the polymer in the agent, all possible physiologically acceptable anions can be used, such as chloride, bromide, hydrogen sulfate, methyl sulfate, ethyl sulfate, tetrafluoroborate, phosphate, hydrogen phosphate, dihydrogen phosphate or p-toluene sulfonate, or triflate.

Exemplary inventive (C₁ to C₄) alkyl groups are methyl, ethyl, propyl, isopropyl, butyl, sec-butyl, isobutyl, and tert-butyl.

Exemplary inventive (C₈ to C₃₀) alkyl groups are octyl(capryl), decyl(caprinyl), dodecyl(lauryl), tetradecyl(myristyl), hexadecyl(cetyl), octadecyl(stearyl), eicosyl(arachyl), and docosyl(behenyl).

Molecular weights of amphiphilic, cationic polymers according to the invention are preferably from 10,000 g/mol to 50,000,000 g/mol, more preferably from 50,000 g/mol to 5,000,000 g/mol, and particularly preferably from 75,000 g/mol to 1,000,000 g/mol.

In the context of the invention, preferred agents contain amphiphilic, cationic polymers (a) in an amount of 0.1 wt. % to 20.0 wt. %, more preferably 0.2 wt. % to 10.0 wt. %, and quite preferably 0.5 wt. % to 7.0 wt. %, based on total weight of the agent.

Properties of the agent according to the invention have proven to be particularly advantageous when the agent is packaged as an aerosol spray, aerosol foam, pump spray or pump foam. This preferred packaging form is described later in detail.

The following amphiphilic, cationic polymers (a) are inventively preferably employed in the agents when the amphiphilic, cationic polymers (a) corresponding to the above Formulas (I) to (IV) fulfill one or more of the following criteria:
R¹ and R⁴ are each a methyl group,
X¹ is an NH group,
X² is an NH group,
A¹ and A² are, independently of one another, ethane-1,2-diyl or propane-1,3-diyl,
R², R³, R⁵ and R⁶ are, independently of one another, methyl or ethyl, (preferably methyl), and
R⁷ is a (C₁₀ to C₂₄) alkyl group, especially decyl(caprinyl), dodecyl(lauryl), tetradecyl(myristyl), hexadecyl(cetyl), octadecyl(stearyl), eicosyl(arachyl) or docosyl(behenyl).

Preferably, the structural unit of Formula (III) is chosen from at least one of the structural units of Formulae (III-1) to (III-8)

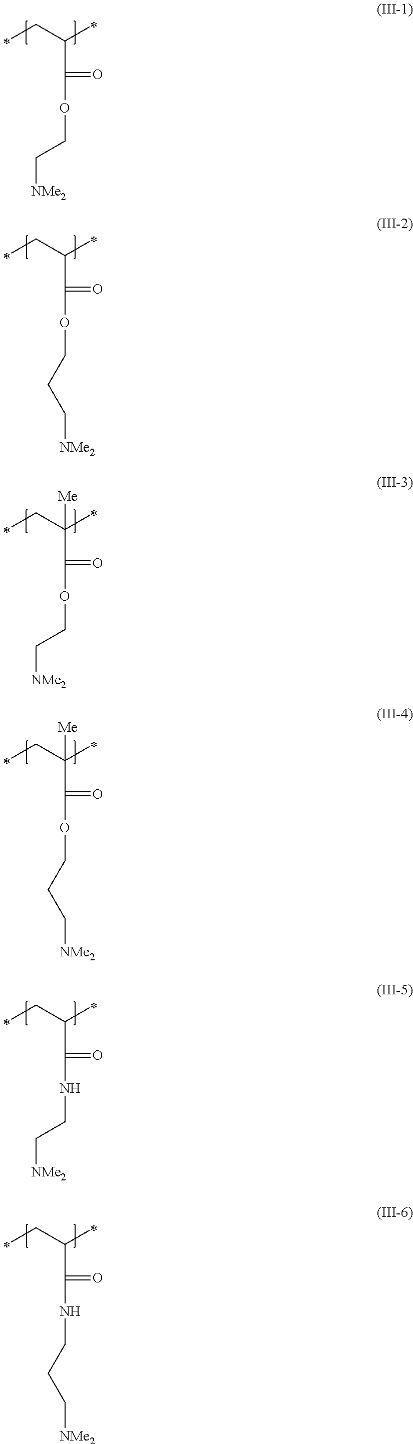

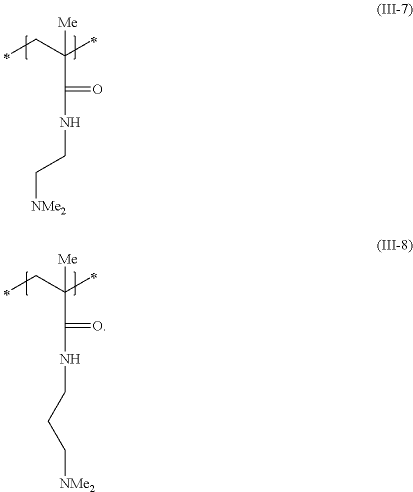

Moreover, it proves particularly preferable to select the structural unit according to Formula (III-7) and/or of Formula (III-8) as the structural unit of Formula (III). According to the invention, the structural unit of Formula (III-8) is a quite particularly preferred structural unit.

Furthermore, the structural unit of Formula (IV) is preferably chosen from at least one structural unit of Formulae (IV-1) to (IV-8)

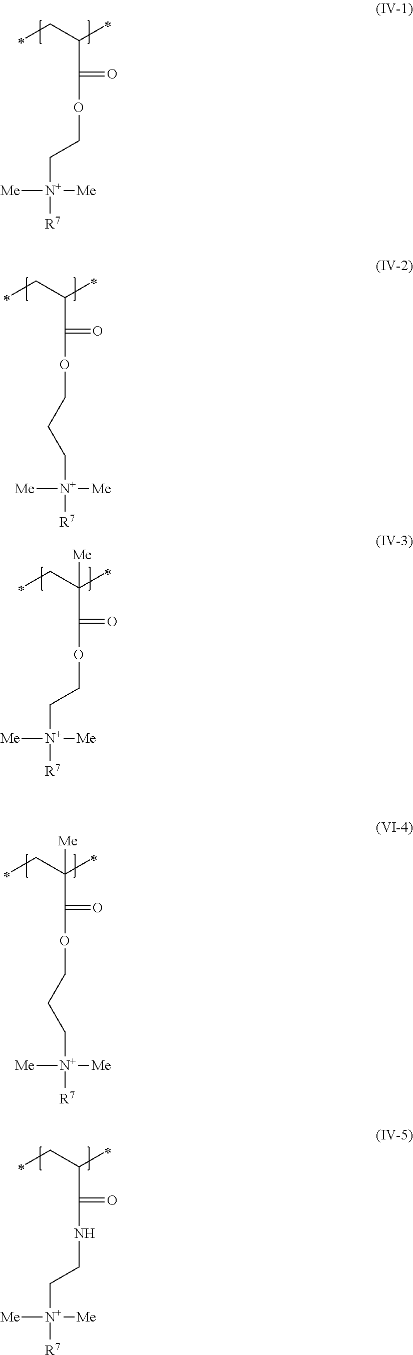

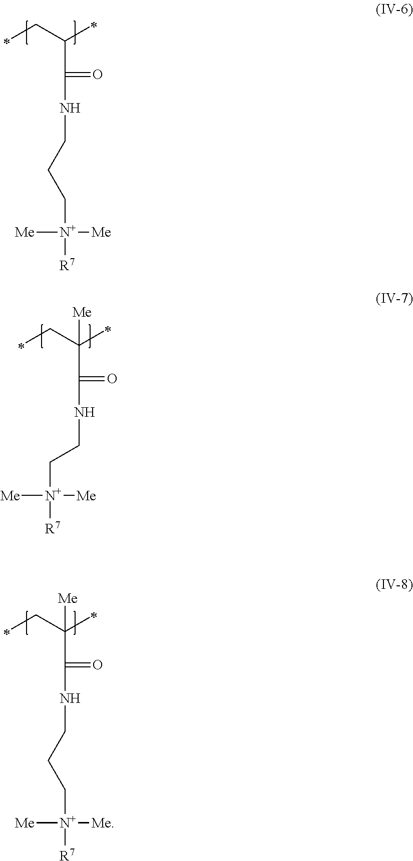

wherein each R⁷ is a (C₈ to C₃₀) alkyl group.

The structural units of Formula (IV-7) and/or of Formula (IV-8) are once again particularly preferred as the structural unit of Formula (IV), wherein each R⁷ is octyl(capryl), decyl(caprinyl), dodecyl(lauryl), tetradecyl(myristyl), hexadecyl(cetyl), octadecyl(stearyl), eicosyl(arachyl) oder docosyl(behenyl). According to the invention, the structural unit of Formula (IV-8) represents a quite particularly preferred structural unit of Formula (IV).

An amphiphilic, cationic polymer having at least one structural unit of Formula (I), at least one structural unit of Formula (II), at least one structural unit of Formula (III-8), and at least one structural unit of Formula (IV-8) is quite particularly preferably present in the agent according to the invention,

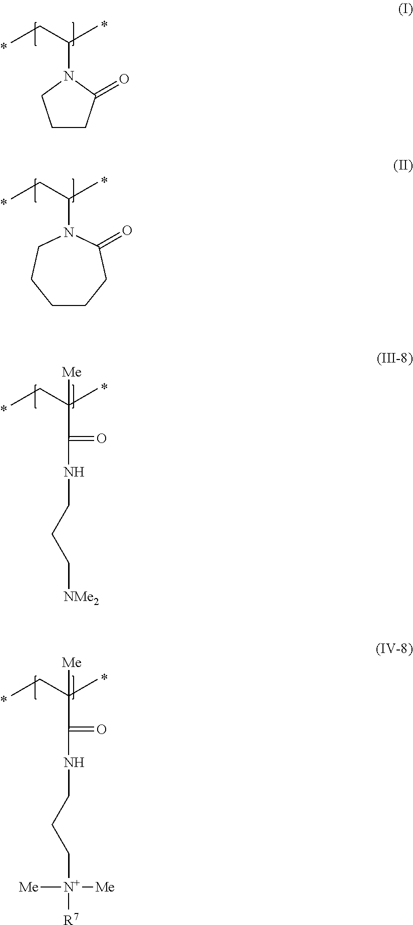

wherein R⁷ is octyl(capryl), decyl(caprinyl), dodecyl(lauryl), tetradecyl(myristyl), hexadecyl(cetyl), octadecyl(stearyl), eicosyl(arachyl) or docosyl(behenyl).

A quite particularly preferred amphiphilic, cationic polymer is the copolymer of N-vinyl pyrrolidone, N-vinyl caprolactam, N-(3-dimethylaminopropyl)methacrylamide and 3-(methacryloylamino)propyl-lauryl-dimethylammonium chloride (INCI name: Polyquaternium-69), marketed, for example, under the trade name AquaStyle® 300 (28-32 wt. % active substance in an ethanol-water mixture, molecular weight 350 000) by the ISP company.

Additionally, the agent has at least one film-forming and/or setting polymer chosen from chitosan and its derivatives.

Chitosans are biopolymers and are considered to be in the hydrocolloid group. From a chemical point of view, they are partially deacetylated chitins of different molecular weight.

Chitosan is manufactured from chitin, preferably from the remains of crustacean shells, which are available in large quantities as a cheap raw material. For this, the chitin is firstly deproteinated by adding bases, demineralized by adding mineral acids and finally deacetylated by adding strong bases, wherein the molecular weights can vary over a wide spectrum. Those types are preferably employed that have an average molecular weight of 800,000 to 1,200,000 Dalton, a viscosity according to Brookfield (1 wt. % conc. in glycolic acid) of 5000 mPas or less, a deacetylation degree in the range 80 to 88% and an ash content of 0.3 wt. % or less.

In addition to chitosans as typical cationic biopolymers, cationically derivatized chitosans can also be considered (such as quaternized products) or alkoxylated chitosans.

Inventively preferred agents contain neutralization products of chitosan with at least one acid as the chitosan derivative(s) according to the invention. The acid utilized for neutralization is preferably chosen from organic acids, particularly from formic acid, acetic acid, citric acid, lactic acid, pyrrolidone carboxylic acid, nicotinic acid, hydroxy-isobutyric acid, hydroxy-isovalearic acid, or mixtures of these acids. It is quite particularly preferred to choose the acid(s) utilized for neutralization from lactic acid, pyrrolidone carboxylic acid, nicotinic acid, hydroxy-isobutyric acid, hydroxy-isovalearic acid or mixtures of these acids.

Exemplary suitable chitosan (derivatives) are freely available on the market under the trade names Hydagen® CMF (1 wt. % active substance in aqueous solution with 0.4 wt. % glycolic acid, molecular weight 500,000 to 5,000,000 g/mol Cognis), Hydagen® HCMF (chitosan (80% deacetylated), molecular weight 50,000 to 1 000 000 g/mol, Cognis), Kytamer® PC (80 wt. % active substance of Chitosan pyrrolidone carboxylate (INCI name: Chitosan PCA), Amerchol) and Chitolam® NB/101.

The agents preferably contain chitosan or its derivatives in an amount of 0.01 wt. % to 20.0 wt. %, more preferably 0.01 wt. % to 10.0 wt. %, and quite preferably 0.1 wt. % to 1 wt. %, based on total weight of the agent.

A preferred embodiment of the agent according to the invention is an agent for treating keratin-containing fibers, especially human hair, comprising in a cosmetically acceptable carrier
(a) at least one amphiphilic, cationic polymer having at least one structural unit of Formula (I), at least one structural unit of Formula (II), at least one structural unit of Formula (III-8), and at least one structural unit of Formula (IV-8),

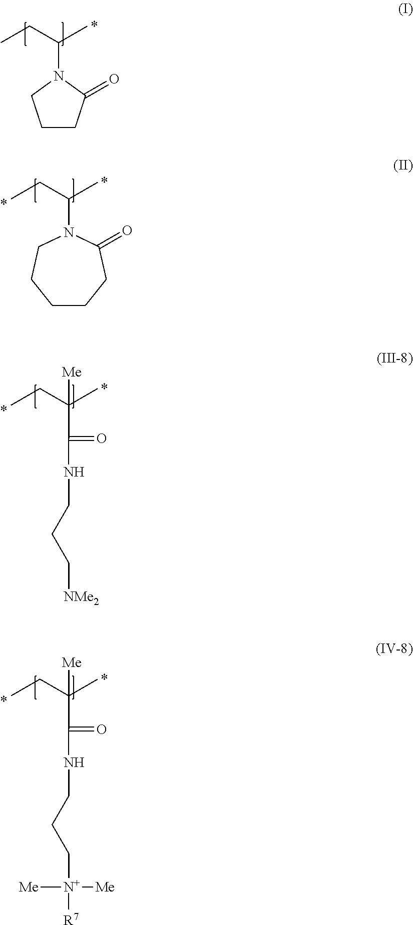

wherein R⁷ is octyl(capryl), decyl(caprinyl), dodecyl(lauryl), tetradecyl(myristyl), hexadecyl(cetyl), octadecyl(stearyl), eicosyl(arachyl) or docosyl(behenyl), and
(b) at least one film-forming and/or setting polymer chosen from a neutralization product of chitosan with at least one organic acid.

In particular, the previously mentioned polymer with the INCI name Polyquaternium 69 is preferred as the amphiphilic cationic polymer (see above).

In the context of the above embodiment, the neutralization products of chitosan are once again preferably chosen from neutralization products of chitosan with at least one organic acid chosen from lactic acid, pyrrolidone carboxylic acid, nicotinic acid, hydroxy-isobutyric acid, hydroxy-isovalearic acid or mixtures of these acids.

Combinations of the above amphiphilic, cationic polymer (a) with at least one neutralization product of chitosan chosen from Hydagen® CMF (1 wt. % active substance in aqueous solution with 0.4 wt. % glycolic acid, molecular weight 500,000 to 5,000,000 g/mol Cognis), Hydagen® HCMF (chitosan (80% deacetylated), molecular weight 50,000 to 1,000,000 g/mol, Cognis), Kytamer® PC (80 wt. % active substance of chitosan pyrrolidone carboxylate (INCI name: Chitosan PCA), Amerchol), Chitolam® NB/101 or mixtures thereof, are particularly suitable.

Similarly, the previously mentioned quantitative data are also preferred mutatis mutandis for this embodiment.

In addition to the incorporated chitosan (derivative), the agents can have a further film-forming and/or setting polymer chosen from non-ionic polymers and/or from permanently cationic polymers and/or from amphoteric polymers.

According to a specific embodiment, the agent includes at least one film-forming non-ionic and/or setting non-ionic polymer (c1) in addition to polymers (a) and (b).

Preferably, the film-forming non-ionic and/or setting non-ionic polymer (c1) is chosen from at least one film-forming non-ionic and/or setting non-ionic polymer having at least one structural unit chosen from the structural units of Formulae (M1) to (M3)

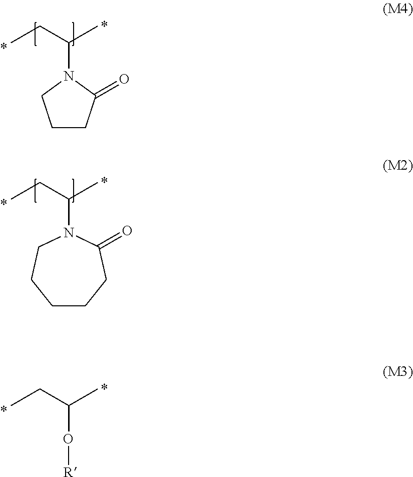

wherein R′ is a hydrogen atom or a (C₂ to C₁₈) acyl group.

Inventively preferred suitable film-forming non-ionic and/or setting non-ionic polymers (c1) containing at least one structural element of Formula (M3) carry a hydrogen atom, an acetyl group or a propanoyl group, especially a hydrogen atom or an acetyl group as R′.

The film-forming non-ionic and/or setting non-ionic polymers (c1) are again preferably chosen from at least one polymer of the group of
homopolymers and non-ionic copolymers of N-vinyl pyrrolidone,
polyvinyl alcohol, and
polyvinyl acetate.

Suitable polyvinyl pyrrolidones include commercial products such as Luviskol® K 90 or Luviskol® K 85 from BASF SE.

Suitable polyvinyl alcohols are marketed, for example, under the trade names Elvanol® by Du Pont or Vinol® 523/540 by Air Products.

A suitable polyvinyl acetate is marketed, for example, as an emulsion under the trade name Vinac® by Air Products.

Agents containing as the film-forming non-ionic and/or setting non-ionic polymer (c1) at least one polymer chosen from the group of
polyvinyl pyrrolidone, and
copolymers of N-vinyl pyrrolidone and vinyl esters of carboxylic acids containing 2 to 18 carbon atoms, especially N-vinyl pyrrolidone and vinyl acetate,
   are inventively quite particularly preferred.

Accordingly, those agents are particularly preferred that have in a cosmetically acceptable carrier
(a) at least one amphiphilic, cationic polymer having at least one structural unit of Formula (I), at least one structural unit of Formula (II), at least one structural unit of Formula (III-8), and at least one structural unit of Formula (IV-8),

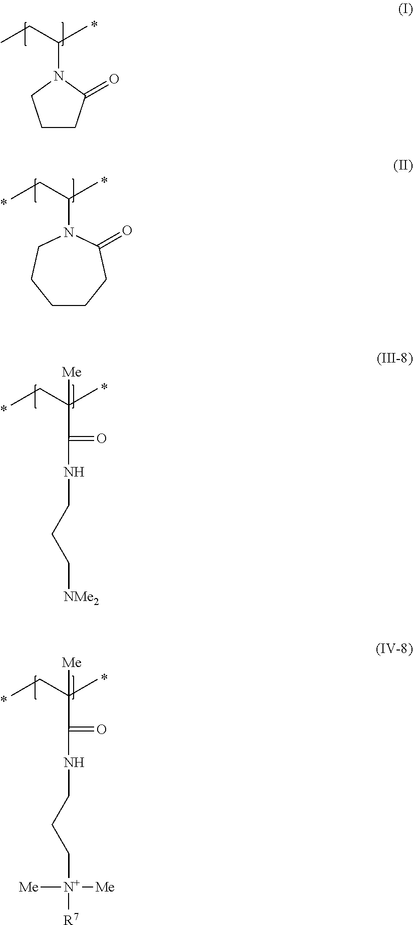

wherein R⁷ is octyl(capryl), decyl(caprinyl), dodecyl(lauryl), tetradecyl(myristyl), hexadecyl(cetyl), octadecyl(stearyl), eicosyl(arachyl) or docosyl(behenyl),
(b) at least one film-forming and/or setting polymer chosen from chitosan and its derivatives, and
(c1) polyvinyl pyrrolidone.

Those agents are quite particularly preferred that have in a cosmetically acceptable carrier
(a) at least one amphiphilic, cationic polymer having at least one structural unit of Formula (I), at least one structural unit of Formula (II), at least one structural unit of Formula (III-8) and at least one structural unit of Formula (IV-8),

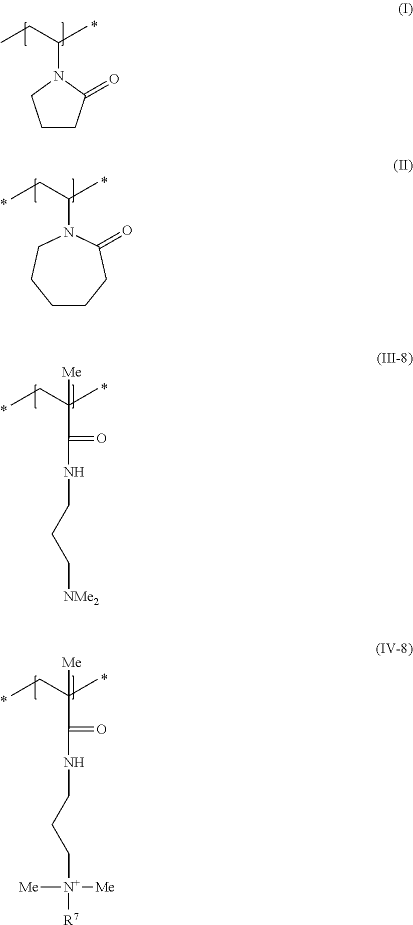

wherein R⁷ is octyl(capryl), decyl(caprinyl), dodecyl(lauryl), tetradecyl(myristyl), hexadecyl(cetyl), octadecyl(stearyl), eicosyl(arachyl) or docosyl(behenyl),
(b) at least one film-forming and/or setting polymer chosen from chitosan and its derivatives, and
(c1) a copolymer manufactured from the monomers N-vinyl pyrrolidone and vinyl acetate, particularly from no additional monomers.

Accordingly, it is again preferred when the molar ratio of the structural units of the monomer N-vinyl pyrrolidone to the structural units of the monomer vinyl acetate of the polymer is in the range of 20 to 80 to 80 to 20, in particular 30 to 70 to 70 to 30.

Suitable copolymers of vinyl pyrrolidone and vinyl acetate are available, for example, under the trade names Luviskol® VA 37, Luviskol® VA 55, Luviskol® VA 64 and Luviskol® VA 73 from BASF SE.

In the context of this embodiment, the previously cited preferred embodiments of the amphiphilic, cationic polymer (a) are preferred (see above). Similarly, all previously mentioned quantitative data regarding polymer components (a) and (b) of the agent are also preferred mutatis mutandis for these embodiments.

According to a specific embodiment, the agent can have, in addition to polymers (a) and (b), at least one film-forming permanently cationic and/or setting permanently cationic polymer (c2) having at least one structural unit that has at least one permanently cationized nitrogen atom. These polymers (c2) differ from the amphiphilic, cationic polymers (a).

“Permanently cationized nitrogen atoms” refer to those nitrogen atoms having a positive charge and thereby form a quaternary ammonium compound. Quaternary ammonium compounds are mostly produced by reacting tertiary amines with alkylating agents, such as methyl chloride, benzyl chloride, dimethyl sulfate, dodecyl bromide, as well as ethylene oxide. Depending on the tertiary amine, the following groups are particularly well known: alkylammonium compounds, alkenylammonium compounds, imidazolinium compounds and pyridinium compounds.

Preferably, the agents include the film-forming, cationic and/or setting cationic polymers (c2) in an amount of 0.1 wt. % to 20.0 wt. %, more preferably 0.2 wt. % to 10.0 wt. %, quite preferably 0.5 wt. % to 5.0 wt. %, based on total weight of the agent.

According to the invention, cationic film-forming and/or cationic setting polymers are preferably chosen from cationic, quatemized cellulose derivatives.

Those agents are again particularly suitable that have in a cosmetically acceptable carrier
(a) at least one amphiphilic, cationic polymer having at least one structural unit of Formula (I), at least one structural unit of Formula (II), at least one structural unit of Formula (III), and at least one structural unit of Formula (IV),

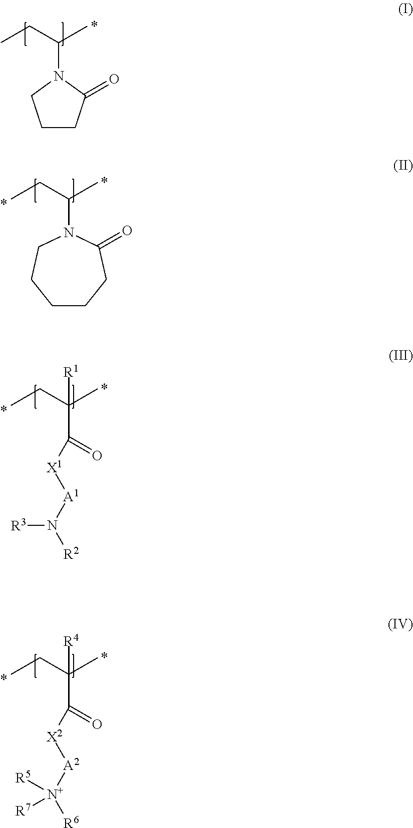

wherein
R¹ and R⁴ are, independently of one another, a hydrogen atom or a methyl group,
X¹ and X² are, independently of one another, an oxygen atom or an NH group,
A¹ and A² are, independently of one another, an ethane-1,2-diyl, propane-1,3-diyl or butane-1,4-diyl group,
R², R³, R⁵ and R⁶ are, independently of one another, a (C₁ to C₄) alkyl group,
R⁷ is a (C₈ to C₃₀) alkyl group,
(b) at least one film-forming and/or setting polymer chosen from chitosan and its derivatives, and
(c2) at least one additional film-forming cationic and/or setting cationic polymer chosen from cationic, quaternized cellulose derivatives.

Those agents are particularly suitable that have in a cosmetically acceptable carrier
(a) at least one amphiphilic, cationic polymer having at least one structural unit of Formula (I), at least one structural unit of Formula (II), at least one structural unit of Formula (III-8), and at least one structural unit of Formula (IV-8),

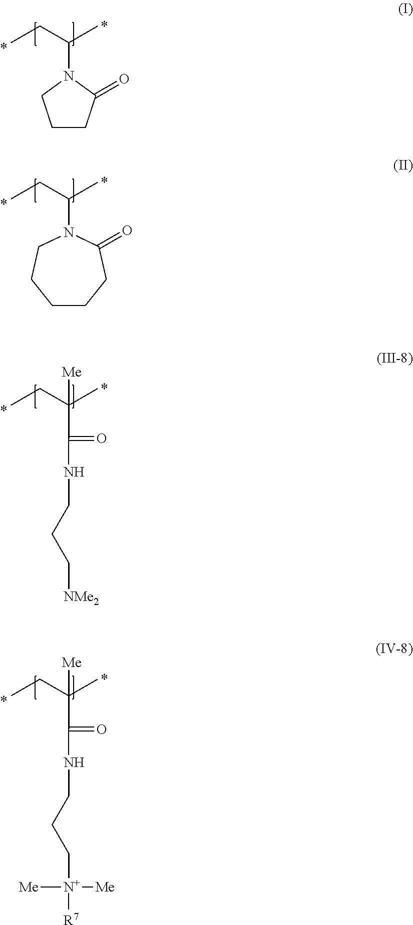

wherein R⁷ is octyl(capryl), decyl(caprinyl), dodecyl(lauryl), tetradecyl(myristyl), hexadecyl(cetyl), octadecyl(stearyl), eicosyl(arachyl) or docosyl(behenyl),
(b) at least one film-forming and/or setting polymer chosen from chitosan and its derivatives, and
(c2) at least one additional film-forming cationic and/or setting cationic polymer chosen from cationic, quaternized cellulose derivatives.

In general, those cationic, quaternized celluloses having more than one permanent cationic charge in a side chain have proven to be advantageous.

Among the cationic cellulose derivatives to be highlighted are those produced from the reaction of hydroxyethyl cellulose with a dimethyldiallylammonium reactant (especially dimethyldiallylammonium chloride), optionally in the presence of further reactants. Among these cationic celluloses, once again those cationic celluloses with the INCI name Polyquaternium-4 are particularly suitable, marketed, for example, by the National Starch company under the trade names Celquat® H 100, Celquat® L 200.

Consequently, agents according to the invention are particularly suitable that have in a cosmetically acceptable carrier
(a) at least one amphiphilic, cationic polymer having at least one structural unit of Formula (I), at least one structural unit of Formula (II), at least one structural unit of Formula (III-8), and at least one structural unit of Formula (IV-8)

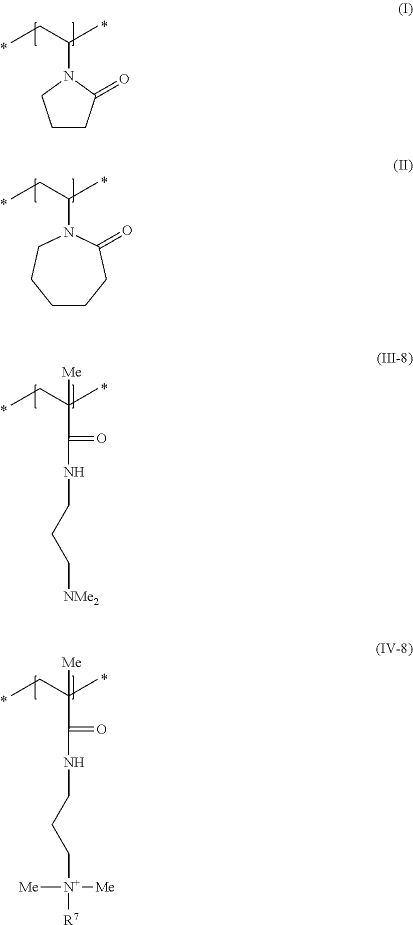

wherein R⁷ is octyl(capryl), decyl(caprinyl), dodecyl(lauryl), tetradecyl(myristyl), hexadecyl(cetyl), octadecyl(stearyl), eicosyl(arachyl) or docosyl(behenyl),
(b) at least one film-forming and/or setting polymer chosen from chitosan and its derivatives and
(c2) at least one additional film-forming cationic and/or setting cationic polymer chosen from cationic, quaternized cellulose derivatives produced from the reaction of hydroxyethyl cellulose with a dimethyldiallylammonium reactant (especially dimethyldiallylammonium chloride), optionally in the presence of further reactants.

In the context of these abovementioned embodiments, the previously cited preferred embodiments of amphiphilic, cationic polymer (a) are suitable (see above). Similarly, all previously mentioned preferred quantitative data regarding polymer components (a) and (b) of the agent are also well suited mutatis mutandis for these embodiments.

In addition, at least one film-forming cationic and/or setting cationic polymer (c2) having at least one structural element of Formula (I), at least one structural element of Formula (VI), and optionally at least one structural element of Formula (V) is particularly preferably suitable

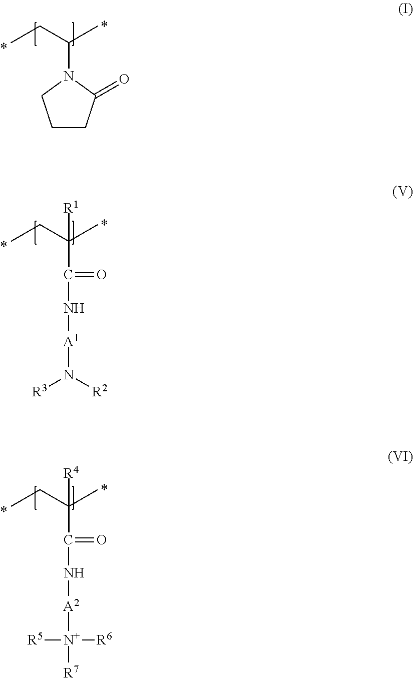

wherein
R¹ and R⁴ are, independently of one another, a hydrogen atom or a methyl group,
A¹ and A² are, independently of one another, an ethane-1,2-diyl, propane-1,3-diyl or butane-1,4-diyl group,
R², R³, R⁵ and R⁶ are, independently of one another, a (C₁ to C₄) alkyl group,
R⁷ is a (C₈ to C₃₀) alkyl group.

To compensate for the positive charge of the monomer (VI), all possible physiologically acceptable anions can be used, such as chloride, bromide, hydrogen sulfate, methyl sulfate, ethyl sulfate, tetrafluoroborate, phosphate, hydrogen phosphate, dihydrogen phosphate or p-toluene sulfonate, or triflate.

Accordingly, those agents are particularly suitable that have in a cosmetically acceptable carrier
(a) at least one amphiphilic, cationic polymer having at least one structural unit of Formula (I), at least one structural unit of Formula (II), at least one structural unit of Formula (III), and at least one structural unit of Formula (IV),

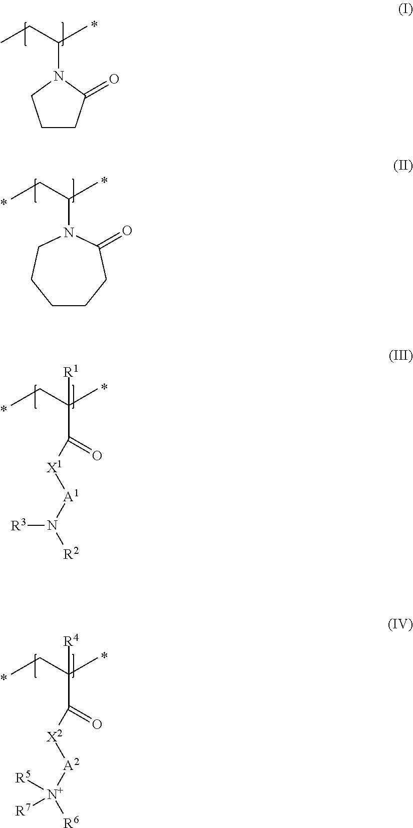

wherein
R¹ and R⁴ are, independently of one another, a hydrogen atom or a methyl group,
X¹ and X² are, independently of one another, an oxygen atom or an NH group,
A¹ and A² are, independently of one another, an ethane-1,2-diyl, propane-1,3-diyl or butane-1,4-diyl group,
R², R³, R⁵ and R⁶ are, independently of one another, a (C1 to C₄) alkyl group, and
R⁷ is a (C₈ to C₃₀) alkyl group,
(b) at least one film-forming and/or setting polymer chosen from chitosan and its derivatives and p0 (c2) at least one additional cationic film-forming and/or cationic setting polymer having at least one structural unit of Formula (I), at least one structural unit of Formula (VI), and at least one structural unit of Formula (V)

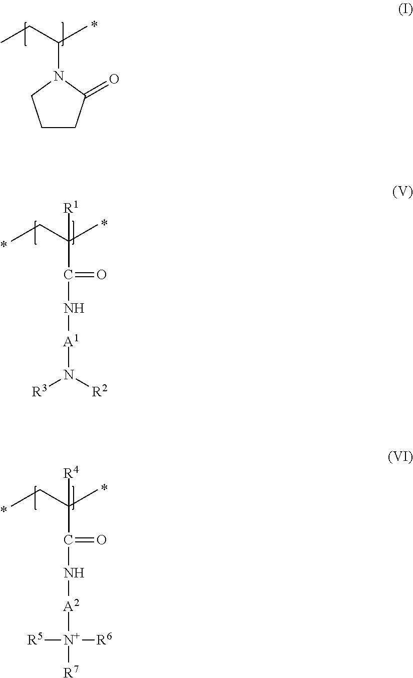

in which
R¹ and R⁴ are, independently of one another, a hydrogen atom or a methyl group,
A¹ and A² are, independently of one another, an ethane-1,2-diyl, propane-1,3-diyl or butane-1,4-diyl group,
R², R³, R⁵ and R⁶ are, independently of one another, a (C1 to C₄) alkyl group,
R⁷ is a (C₈ to C₃₀) alkyl group.

Moreover, those agents are particularly preferred that have in a cosmetically acceptable carrier
(a) at least one amphiphilic, cationic polymer having at least one structural unit of Formula (I), at least one structural unit of Formula (II), at least one structural unit of Formula (III-8), and at least one structural unit of Formula (IV-8)

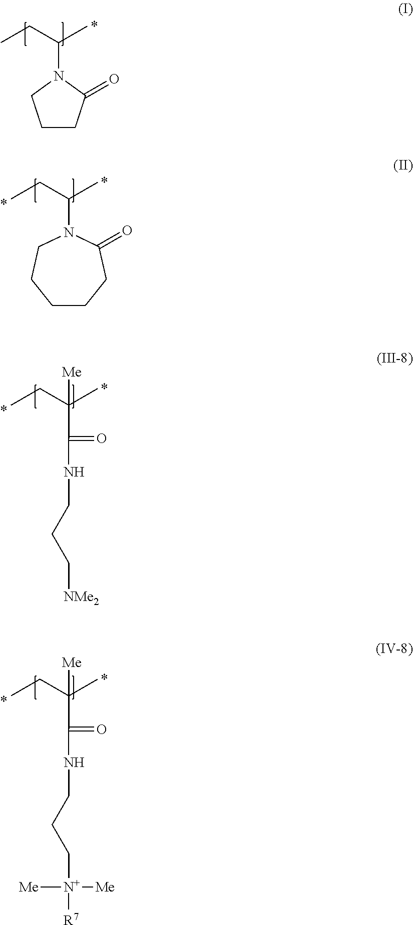

wherein R⁷ is octyl(capryl), decyl(caprinyl), dodecyl(lauryl), tetradecyl(myristyl), hexadecyl(cetyl), octadecyl(stearyl), eicosyl(arachyl) or docosyl(behenyl),
(b) at least one film-forming and/or setting polymer selected from chitosan and its derivatives and
(c2) at least one additional cationic film-forming and/or cationic setting polymer having at least one structural unit of Formula (I), at least one structural unit of Formula (VI), and at least one structural unit of Formula (V)

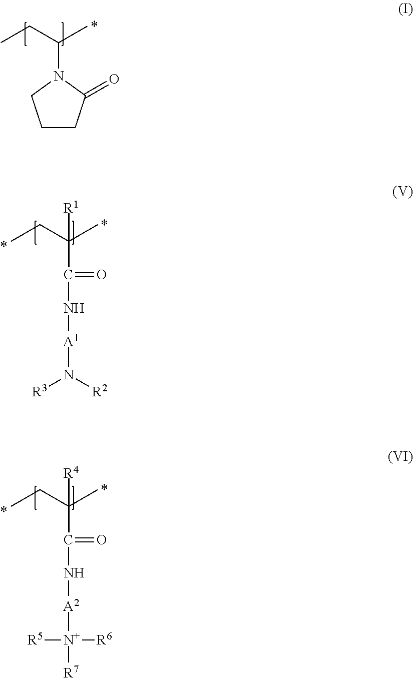

wherein
R¹ and R⁴ are, independently of one another, a hydrogen atom or a methyl group,
A¹ and A² are, independently of one another, an ethane-1,2-diyl, propane-1,3-diyl or butane-1,4-diyl group,
R², R³, R⁵ and R⁶ are, independently of one another, a (C₁ to C₄) alkyl group, and
R⁷ is a (C₈ to C₃₀) alkyl group.

To compensate the positive charge of the monomer (VI), the above relevant statement applies.

Exemplary suitable compounds (c2) include
copolymers of dimethylaminoethyl methacrylate, quaternized with diethyl sulfate, with vinyl pyrrolidone having the INCI name Polyquatemium-11 under the trade names Gafquat® 440, Gafquat® 734, Gafquat® 755 (each from ISP) and Luviquat PQ 11 PN (BASF SE), and
copolymers of methacryloylaminopropyllauryldimethylammonium chloride with vinyl pyrrolidone and dimethylaminopropylmethacrylamide with the INCI name Polyquaternium-55, commercially available under the trade names, Styleze® W-10, Styleze® W-20 (ISP).

In the context of this embodiment, the previously cited preferred embodiments of the amphiphilic, cationic polymer (a) are preferred (see above). Similarly, all previously mentioned quantitative data regarding polymer components (a) and (b) of the agent are also preferred mutatis mutandis for these embodiments.

In the context of the invention, those cationic film-forming and/or cationic setting copolymers (c2) having at least one structural element of Formula (M4) likewise serve as a preferred employable permanently cationic polymer (c2)

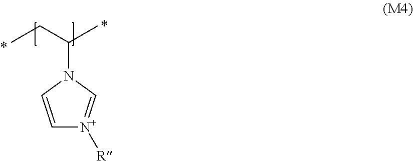

wherein R″ is a (C₁ to C₄) alkyl group, particularly a methyl group, and
additionally has at least one further cationic and/or non-ionic structural element.

Accordingly, those agents are preferred that have in a cosmetically acceptable carrier
(a) at least one amphiphilic, cationic polymer having at least one structural unit of Formula (I), at least one structural unit of Formula (II), at least one structural unit of Formula (III), and at least one structural unit of Formula (IV),

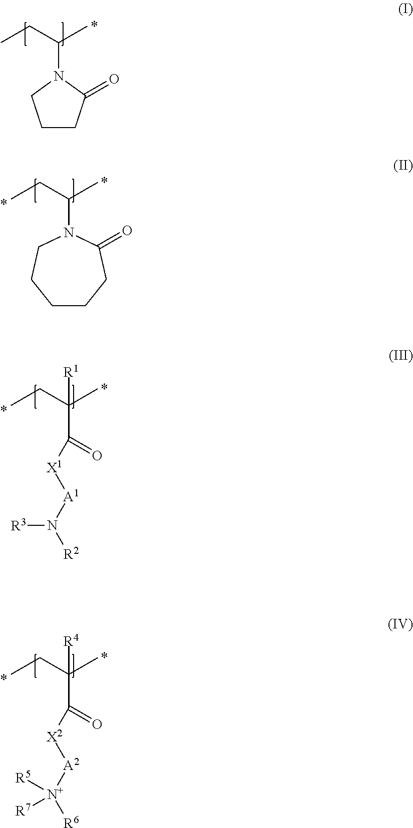

wherein
R¹ and R⁴ are, independently of one another, a hydrogen atom or a methyl group,
X¹ and X² are, independently of one another, an oxygen atom or an NH group,
A¹ and A² are, independently of one another, an ethane-1,2-diyl, propane-1,3-diyl or butane-1,4-diyl group,
R², R³, R⁵ and R⁶ are, independently of one another, a (C₁ to C₄) alkyl group,
R⁷ is a (C₃ to C₃₀) alkyl group,
(b) at least one film-forming and/or setting polymer chosen from chitosan and its derivatives and
(c2) at least one additional cationic film-forming and/or cationic setting polymer having at least one structural element of Formula (M4) as the at least one structural unit having a permanently cationized nitrogen atom

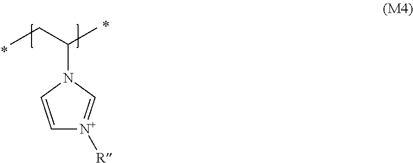

wherein R″ is a (C₁ to C₄) alkyl group, particularly a methyl group, and
additionally having at least one further cationic and/or non-ionic structural element.

Agents corresponding to this embodiment form transparent (i.e., non-turbid) compositions. Any turbidity cannot be seen with the naked eye. Moreover, the agents additionally possess to an excellent degree the advantageous parameters regarding the strong degree of hold for the hairstyle or volume or for haircare.

To compensate for the positive polymer charge of the component (c2), all possible physiologically acceptable anions can be used, such as chloride, bromide, hydrogen sulfate, methyl sulfate, ethyl sulfate, tetrafluoroborate, phosphate, hydrogen phosphate, dihydrogen phosphate or p-toluene sulfonate, or triflate.

Those agents are particularly preferred that have in a cosmetically acceptable carrier
(a) at least one amphiphilic, cationic polymer having at least one structural unit of Formula (I), at least one structural unit of Formula (II), at least one structural unit of Formula (III-8), and at least one structural unit of Formula (IV-8)

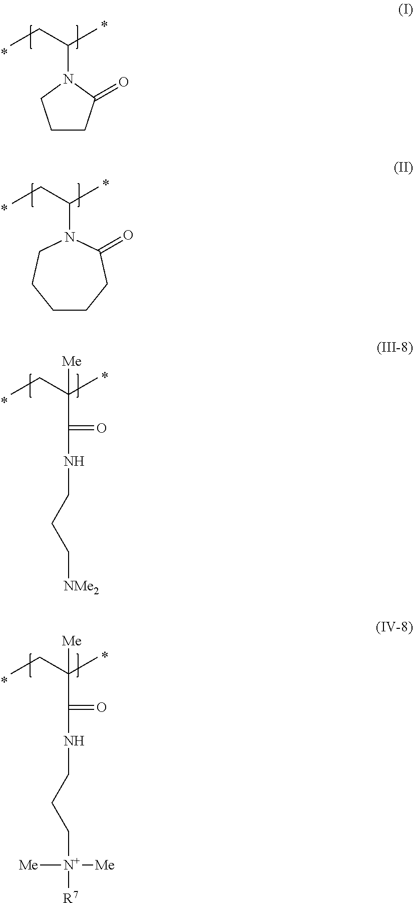

wherein R⁷ is octyl(capryl), decyl(caprinyl), dodecyl(lauryl), tetradecyl(myristyl), hexadecyl(cetyl), octadecyl(stearyl), eicosyl(arachyl) or docosyl(behenyl),
(b) at least one film-forming and/or setting polymer chosen from chitosan and its derivatives and
(c2) at least one cationic film-forming and/or cationic setting polymer having at least one structural element of Formula (M4)

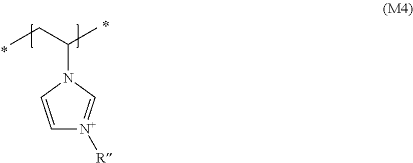

wherein R″ is a (C₁ to C₄) alkyl group, particularly a methyl group, and
additionally possesses at least one further cationic and/or non-ionic structural element.

In particular, the previously mentioned polymer with INCI name Polyquaternium 69 is particularly preferred as the amphiphilic cationic polymer (a) (see above).

To compensate the positive polymer charge of component (c2), the above relevant statement applies.

It is again inventively preferred when, in addition to amphiphilic cationic polymer (a) as the cationic film-forming and/or cationic setting polymer (c2) of this embodiment, the agent has at least one copolymer (c2-1) that, in addition to a structural element of Formula (M4), further contains a structural element of Formula (I)

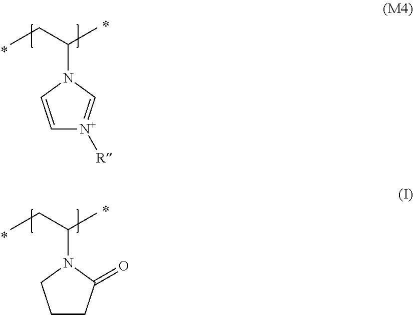

wherein R″ is a (C₁ to C₄) alkyl group, particularly a methyl group.

To compensate for the positive polymer charge of the copolymer (c2-1), all possible physiologically acceptable anions can be used, such as chloride, bromide, hydrogen sulfate, methyl sulfate, ethyl sulfate, tetrafluoroborate, phosphate, hydrogen phosphate, dihydrogen phosphate or p-toluene sulfonate, or triflate.

Quite particularly preferred cationic film-forming and/or cationic setting polymers as copolymers (c2-1) contain 10 to 30 mol %, preferably 15 to 25 mol %, and particularly 20 mol % of structural units according to Formula (M4), and 70 to 90 mol %, preferably 75 to 85 mol % and particularly 80 mol % of structural units according to Formula (I).

It is particularly preferred when copolymers (c2-1) have, in addition to polymer units resulting from the incorporation of the cited structural units according to Formula (M4) and (I) into the copolymer, a maximum of 5 wt. %, preferably a maximum of 1 wt. % of polymer units that trace back to the incorporation of other monomers. Copolymers (c2-1) are preferably exclusively formed from structural units of Formula (M4) with R″=methyl and (I) and can be described by the general Formula (Polyl)

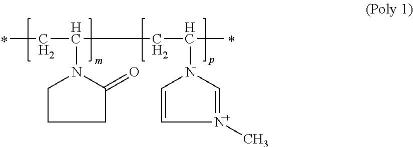

wherein indices m and p vary according to the molecular mass of the polymer and are not intended to mean to portray block copolymers. In fact, structural units of Formula (M4) and Formula (I) can be statistically distributed in the molecule.

If a chloride ion is used to compensate the positive charge of the polymer of Formula (Poly1), then these N-methyl vinylimidazole/vinyl pyrrolidone copolymers are named according to INCI nomenclature as Polyquaternium-16 and are available from for example BASF under the trade names Luviquat® Style, Luviquat® FC 370, Luviquat® FC 550, Luviquat® FC 905 and Luviquat® HM 552.

If a methosulfate ion is used to compensate for the positive charge of the polymer of Formula (Poly1), then these N-methyl vinylimidazole/vinyl pyrrolidone copolymers are named according to INCI nomenclature as Polyquatemium-44 and are available from, for example, BASF under the trade name Luviquat® UltraCare.

Particularly preferred inventive agents contain a copolymer (c2-1), especially according to Formula (Poly1), which has molecular masses within a defined range. Here, inventive agents are preferred in which the molecular mass of copolymer (c2-1) is from 50 to 400 kDa, preferably from 100 to 300 kDa, more preferably from 150 to 250 kDa and particularly from 190 to 210 kDa.

In addition to the copolymer(s) c2-1 or instead of it or them, the inventive agents can also comprise copolymers (c2-2) that starting from the copolymer (c2-1) possess as the additional structural units structural units of Formula (II)

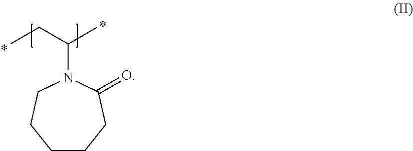

Further particularly preferred agents are those that have as the cationic film-forming and/or cationic setting polymer (c2) at least one copolymer (c2-2) having at least one structural unit according to Formula (M4-a), at least one structural unit according to Formula (I), and at least one structural unit according to Formula (II)

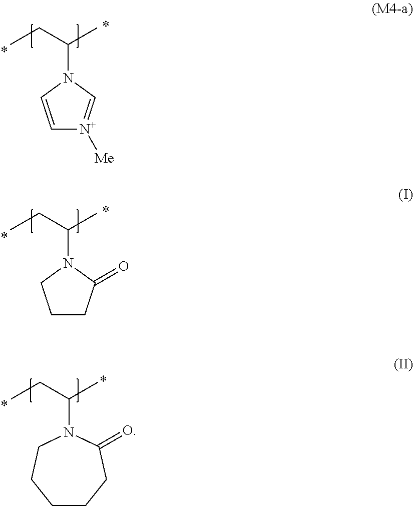

Also in this regard it is particularly preferred when copolymers (c2-2) contain, in addition to polymer units resulting from the incorporation of structural units according to Formula (M4-a), (I) and (II) into the copolymer, a maximum of 5 wt. %, preferably a maximum of 1 wt. % of polymer units that trace back to the incorporation of other monomers. Copolymers (c2-2) are preferably exclusively formed from structural units of Formula (M4-a), (I) and (II) can be described by the general Formula (Poly2)

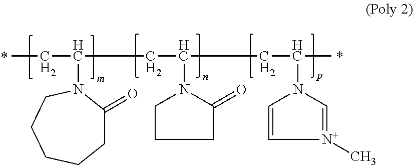

wherein indices m, n and p vary according to the molecular mass of the polymer and are not intended to mean to portray block copolymers. In fact, structural units of the cited Formulas can be statistically distributed in the molecule.

To compensate for the positive polymer charge of the component (c2-2), all possible physiologically acceptable anions can be used, such as chloride, bromide, hydrogen sulfate, methyl sulfate, ethyl sulfate, tetrafluoroborate, phosphate, hydrogen phosphate, dihydrogen phosphate or p-toluene sulfonate, or triflate.

If a methosulfate ion is used to compensate for the positive charge of the polymer of Formula (Poly2), then these N-methyl vinylimidazole/vinyl pyrrolidone/vinyl caprolactam copolymers are named according to INCI nomenclature as Polyquaternium-46 and are available from, for example, BASF under the trade name Luviquat® Hold.

Quite particularly preferred copolymers (c2-2) contain 1 to 20 mol %, preferably 5 to 15 mol % and particularly 10 mol % of structural units according to Formula (M4-a), 30 to 50 mol %, preferably 35 to 45 mol % and particularly 40 mol % of structural units according to Formula (I) and 40 to 60 mol %, preferably 45 to 55 mol % and particularly 60 mol % of structural units according to Formula (II).

Particularly preferred inventive agents include a copolymer (c2-2) having molecular masses within a defined range. Here, inventive agents are preferred wherein the molecular mass of copolymer c2-2 is from 100 to 1000 kDa, preferably from 250 to 900 kDa, more preferably from 500 to 850 kDa and particularly from 650 to 710 kDa.

In addition to copolymer(s) (c2-1) and/or (c2-2) or in its or their place, the agents can also include copolymers (c2-3) as the film-forming cationic and/or setting cationic polymer (c2) which has as structural units structural units of Formulae (M4-a) and (I), as well as structural units from the group of vinyl imidazole units and further structural units from the group of acrylamide and/or methacrylamide units.

Further particularly preferred agents according to the invention comprise as the cationic film-forming and/or cationic setting polymer (c2) at least one copolymer (c2-3) that comprises at least one structural unit according to Formula (M4-a) and at least one structural unit according to Formula (I) and at least one structural unit according to Formula (VI) and at least one structural unit according to Formula (VII)

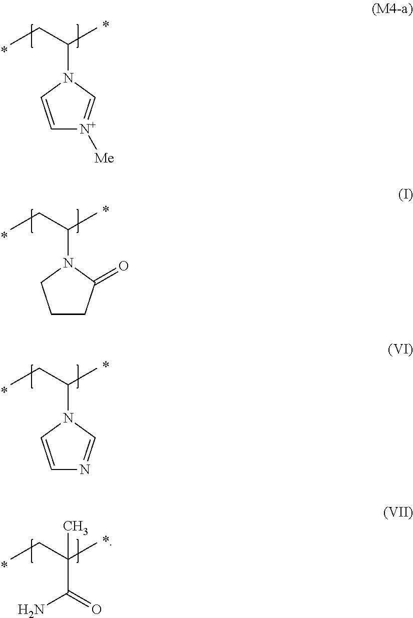

Also in this regard it is particularly preferred when copolymers (c2-3) have, in addition to polymer units resulting from the incorporation of the structural units according to Formula (M4-a), (I), (VI) and (VII) into the copolymer, a maximum of 5 wt. %, preferably a maximum of 1 wt. % of polymer units that trace back to the incorporation of other monomers. Copolymers (c2-3) are preferably exclusively formed from structural units of Formula (M4-a), (I), (VI) and (VII) and can be described by the general Formula (Poly3)

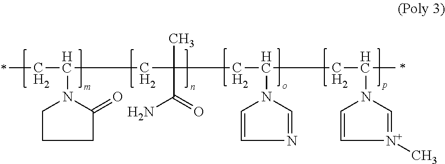

wherein indices m, n, o and p vary according to the molecular mass of the polymer and are not intended to mean to portray block copolymers. In fact, structural units of Formula (M4-a), (I), (VI) and (VII) can be statistically distributed in the molecule.

To compensate for the positive polymer charge of component (c2-3), all possible physiologically acceptable anions can be used, such as chloride, bromide, hydrogen sulfate, methyl sulfate, ethyl sulfate, tetrafluoroborate, phosphate, hydrogen phosphate, dihydrogen phosphate or p-toluene sulfonate, or triflate.

If a methosulfate ion is used to compensate for the positive charge of the polymer of Formula (Poly3), then these N-methyl vinylimidazole/vinyl pyrrolidone/vinyl imidazole/methacrylamide copolymers are named according to INCI nomenclature as Polyquaternium-68 and are available from, for example, BASF under the trade name Luviquat® Supreme.

Particularly preferred copolymers (c2-3) contain 1 to 12 mol %, preferably 3 to 9 mol %, and particularly 6 mol % of structural units according to Formula (M4-a), 45 to 65 mol %, preferably 50 to 60 mol % and particularly 55 mol % of structural units according to Formula (I), 1 to 20 mol %, preferably 5 to 15 mol % and particularly 10 mol % of structural units according to Formula (VI), and 20 to 40 mol %, preferably 25 to 35 mol % and particularly 29 mol % of structural units according to Formula (VII).

Particularly preferred agents contain a copolymer (c2-3) that has molecular masses within a defined range. Here, preferred agents have a molecular mass of copolymer c2-3 from 100 to 500 kDa, preferably from 150 to 400 kDa, more preferably from 250 to 350 kDa, and particularly from 290 to 310 kDa.

Preferred additional film-forming cationic and/or setting polymers chosen from cationic polymers (c2) with at least one structural element of the above Formula (M4) include: vinyl pyrrolidone/1-vinyl-3-methyl-1H-imidazolium chloride copolymers (such as that with the INCI name Polyquaternium-16 sold under the trade names Luviquat® Style, Luviquat® FC 370, Luviquat® FC 550, Luviquat® FC 905 and Luviquat® HM 552 (BASF SE)), vinyl pyrrolidone/1-vinyl-3-methyl-1H-imidazolium methylsulfate copolymers (such as that with the INCI name Polyquaternium-44 sold under the trade name Luviquat® Care (BASF SE)), vinyl pyrrolidone/vinyl caprolactam/1-vinyl-3-methyl-1H-imidazolium terpolymer (such as that with the INCI name Polyquaternium-46 sold under the trade names Luviquat® Care or Luviquat Hold (BASF SE)), vinyl pyrrolidone/methacrylamide/vinyl imidazole/1-vinyl-3-methyl-1H-imidazolium methyl sulfate copolymer (such as that with the INCI name Polyquaternium-68 sold under the trade name Luviquat® Supreme (BASF SE)), as well as mixtures of these polymers.

According to this embodiment, the previously cited preferred embodiments of the amphiphilic, cationic polymer (a) are preferred (see above). Similarly, all previously mentioned quantitative data regarding polymer components (a) and (b) of the agent are also preferred mutatis mutandis for these embodiments.

In order to intensify the effect according to the invention, the agents preferably also have at least one surfactant, with non-ionic, anionic, cationic, ampholytic surfactants being suitable. The group of ampholytic or amphoteric surfactants includes zwitterionic surfactants and ampholytes. According to the invention, the surfactants can already have an emulsifying action.

The agent preferably has additional surfactants in an amount of 0.01 wt. % to 5 wt. %, particularly preferably 0.05 wt. % to 0.5 wt. %, based on total weight of the agent.

It is particularly preferable when the agents have at least one non-ionic surfactant.

Non-ionic surfactants contain, for example, a polyol group, a polyalkylene glycol ether group or a combination of polyol ether groups and polyglycol ether groups as the hydrophilic group.

Exemplary compounds of this type are
addition products of 2 to 100 moles ethylene oxide and/or 1 to 5 moles propylene oxide to linear and branched fatty alcohols containing 8 to 30 carbon atoms, to fatty acids containing 8 to 30 carbon atoms and to alkyl phenols containing 8 to 15 carbon atoms in the alkyl group,
methyl or C₂-C₆ alkyl group end blocked addition products of 2 to 50 moles ethylene oxide and/or 1 to 5 moles propylene oxide to linear and branched fatty alcohols with 8 to 30 carbon atoms, to fatty acids with 8 to 30 carbon atoms and to alkyl phenols with 8 to 15 carbon atoms in the alkyl group, such as, for example, the commercially available types Dehydol® LS, Dehydol® LT (Cognis),
C₁₂-C₃₀ fatty acid mono- and diesters of addition products of 1 to 30 moles ethylene oxide to glycerin,
addition products of 5 to 60 moles ethylene oxide on castor oil and hydrogenated castor oil,
polyol esters of fatty acids, such as the commercial product Hydagen® HSP (Cognis) or Sovermol types (Cognis),
alkoxylated triglycerides,
alkoxylated fatty acid alkyl esters of the formula (E4-I)

R¹CO—(OCH₂CHR²)_{w}OR³ (E4-I)
wherein R¹CO is a linear or branched, saturated and/or unsaturated acyl group containing 6 to 22 carbon atoms, R² is hydrogen or methyl, R³ is linear or branched alkyl groups containing 1 to 4 carbon atoms, and w is a number from 1 to 20,
amine oxides,
mixed hydroxy ethers, such as are described in DE-OS 1 973 8866,
sorbitol esters of fatty acids and addition products of ethylene oxide to sorbitol esters of fatty acids such as the polysorbates,
sugar esters of fatty acids and addition products of ethylene oxide to sugar esters of fatty acids,
addition products of ethylene oxide to fatty acid alkanolamides and fatty amines,
sugar surfactants of the type alkyl and alkenyl oligoglycosides according to Formula (E4-II),

R⁴[G]ₚ (E4-II)
wherein R⁴ is an alkyl or alkenyl group containing 4 to 22 carbon atoms, G is a sugar group containing 5 or 6 carbon atoms, and p is a number from 1 to 10. They can be obtained according to the appropriate methods of preparative organic chemistry.

Alkylene oxide addition products to saturated, linear fatty alcohols and fatty acids, each having 2 to 100 moles ethylene oxide per mole fatty alcohol or fatty acid, are quite particularly preferred non-ionic surfactants. Similarly, preparations with excellent properties are obtained when they have C₁₂-C₃₀ fatty acid mono- and diesters of addition products of 1 to 30 moles ethylene oxide to glycerin and/or addition products of 5 to 60 moles ethylene oxide to castor oil and hydrogenated castor oil as the non-ionic surfactants.

For surfactants represented by the addition products of ethylene oxide and/or propylene oxide to fatty alcohols or derivatives of these addition products, both products with a “normal” homologue distribution as well as those with a narrow homologue distribution may be used. The term “normal” homologue distribution refers to mixtures of homologues obtained from the reaction of fatty alcohols and alkylene oxide using alkali metals, alkali metal hydroxides or alkali metal alcoholates as catalysts. Narrow homologue distributions are obtained if, for example, hydrotalcite, alkaline earth metal salts of ether carboxylic acids, alkaline earth metal oxides, hydroxides or alcoholates are used as catalysts. Use of products with a narrow homologue distribution can be preferred.

Agents according to the invention quite particularly preferably contain as the surfactant at least one addition product of 15 to 100 moles ethylene oxide, especially 15 to 50 moles ethylene oxide on a linear or branched (especially linear) fatty alcohol containing 8 to 22 carbon atoms. These are quite particularly preferably Ceteareth-15, Ceteareth-25 or Ceteareth-50, which are marketed as Eumulgin® CS 15 (COGNIS), Cremophor A25 (BASF SE) or Eumulgin® CS 50 (COGNIS).

Suitable anionic surfactants include all anionic surface-active materials suitable for use on the human body. They have a water solubilizing anionic group such as a carboxylate, sulfate, sulfonate or phosphate group, and a lipophilic alkyl group containing about 8 to 30 carbon atoms. In addition, the molecule can have glycol or polyglycol ether groups, ester, ether and amide groups as well as hydroxyl groups. Exemplary suitable anionic surfactants are, each in the form of the sodium, potassium and ammonium as well as the mono, di and trialkanolammonium salts containing 2 to 4 carbon atoms in the alkanol group, Examples of suitable anionic surfactants, each in the form of the sodium, potassium and ammonium salts as well as the mono-, di- and trialkanolammonium salts with 2 to 4 carbon atoms in the alkanol group, are
linear and branched fatty acids with 8 to 30 carbon atoms (soaps),
ether carboxylic acids of the formula R—O—(CH₂—CH₂O)ₓ—CH₂—COOH, wherein R is a linear alkyl group containing 8 to 30 carbon atoms and x=0 or 1 to 16,
acyl sarcosides with 8 to 24 carbon atoms in the acyl group,
acyl taurides with 8 to 24 carbon atoms in the acyl group,
acyl isethionates with 8 to 24 carbon atoms in the acyl group,
mono- and dialkyl esters of sulfosuccinic acid with 8 to 24 carbon atoms in the alkyl group and mono-alkyl polyoxyethyl esters of sulfosuccinic acid with 8 to 24 carbon atoms in the alkyl group and 1 to 6 oxyethylene groups,
linear alkane sulfonates containing 8 to 24 carbon atoms,
linear alpha-olefin sulfonates containing 8 to 24 carbon atoms,
alpha-sulfo fatty acid methyl esters of fatty acids containing 8 to 30 carbon atoms,
alkyl sulfates and alkyl polyglycol ether sulfates of the Formula R—O(CH₂—CH₂O)ₓ—OSO₃H, wherein R is preferably a linear alkyl group containing 8 to 30 carbon atoms and x=0 or 1 to 12,
mixtures of surface active hydroxyl sulfonates, sulfated hydroxyalkyl polyethylene glycol ethers and/or hydroxyalkylene propylene glycol ethers,
sulfonates of unsaturated fatty acids with 8 to 24 carbon atoms and 1 to 6 double bonds,
esters of tartaric acid and citric acid with alcohols, which represent the addition products of about 2-15 molecules of ethylene oxide and/or propylene oxide on fatty alcohols containing 8 to 22 carbon atoms,
alkyl and/or alkenyl ether phosphates of Formula (E1-I)),

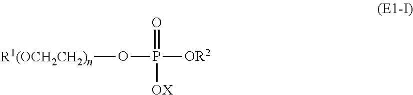

wherein R¹ preferably is an aliphatic hydrocarbon group with 8 to 30 carbon atoms, R² is hydrogen, a group (CH₂CH₂O)ₙR¹ or X, n is a number from 1 to 10, and X is hydrogen, an alkali or alkaline earth metal or NR³R⁴R⁵R⁶, with R³ to R⁶, independently of each other, standing for a C₁ to C₄ hydrocarbon group,
sulfated fatty acid alkylene glycol esters of the formula (E1-II)

R⁷CO(AIkO)ₙSO₃M (E1-II),
wherein R⁷CO— is a linear or branched, aliphatic, saturated and/or unsaturated acyl group with 6 to 22 carbon atoms, Alk is CH₂CH₂, CHCH₃CH₂ and/or CH₂CHCH₃, n is a number from 0.5 to 5, and M is a cation, as described in DE-OS 197 36 906,
monoglyceride sulfates and monoglyceride ether sulfates of Formula (E1-III)

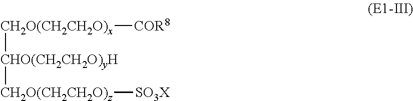

wherein R⁸CO is a linear or branched acyl group containing 6 to 22 carbon atoms, the sum of x, y and z is 0 or a number from 1 to 30, preferably 2 to 10, and X is an alkali metal or alkaline earth metal. In the context of the invention, typical examples of suitable monoglyceride(ether)sulfates are the reaction products of lauric acid monoglyceride, cocoa fatty acid monoglyceride, palmitic acid monoglyceride, stearic acid monoglyceride, oleic acid monoglyceride and tallow fatty acid monoglyceride, as well as their ethylene oxide adducts with sulfur trioxide or chlorosulfonic acid in the form of their sodium salts. Preferably, monoglyceride sulfates of Formula (E1-III) are employed wherein R⁸CO is a linear acyl group containing 8 to 18 carbon atoms,
amido ether carboxylic acids,
condensation products of C₈-C₃₀ fatty alcohols with protein hydrolyzates and/or amino acids and their derivatives, known to one skilled in the art as albumin fatty acid condensates, such as the Lamepon® types, Gluadin® types, Hostapon® KCG or the Amisoft® types.

Preferred anionic surfactants are alkyl sulfates, alkyl polyglycol ether sulfates and ether carboxylic acids with 10 to 18 carbon atoms in the alkyl group and up to 12 glycol ether groups in the molecule, sulfosuccinic acid mono and dialkyl esters with 8 to 18 C atoms in the alkyl group and sulfosuccinic acid mono-alkyl polyoxyethyl esters with 8 to 18 C atoms in the alkyl group and 1 to 6 oxyethylene groups, monoglycerin disulfates, alkyl and alkenyl ether phosphates, as well as albumin fatty acid condensates.

According to the invention, cationic surfactants of the type quaternary ammonium compounds, esterquats and amido amines can likewise be used. Preferred quaternary ammonium compounds are ammonium halides, especially chlorides and bromides, such as alkyltrimethylammonium chlorides, dialkyldimethylammonium chlorides and trialkylmethylammonium chlorides. Long alkyl chains of these surfactants preferably have 10 to 18 carbon atoms, such as in cetyltrimethylammonium chloride, stearyltrimethylammonium chloride, distearyldimethylammonium chloride, lauryldimethylammonium chloride, lauryldimethylbenzylammonium chloride and tricetylmethylammonium chloride. Further preferred cationic surfactants are those imidazolium compounds known under the INCI names Quaternium-27 and Quaternium-83.

Zwitterionic surfactants are surface-active compounds having at least one quaternary ammonium group and at least one —COO⁽⁻⁾ or —SO₃⁽⁻⁾ group in the molecule. Particularly preferred suitable zwitterionic surfactants are betaines such as N-alkyl-N,N-dimethylammonium glycinates, for example, cocoalkyl-dimethylammonium glycinate, N-acyl-aminopropyl-N,N-dimethylammonium glycinate, for example, coco-acylaminopropyl-dimethylammonium glycinate, and 2-alkyl-3-carboxymethyl-3-hydroxyethyl-imidazolines, each with 8 to 18 carbon atoms in the alkyl or acyl group, as well as the cocoacyl-aminoethylhydroxyethylcarboxymethyl glycinate. A preferred zwitterionic surfactant is the fatty acid amide derivative known under the INCI name Cocamidopropyl Betaine.

Ampholytes include such surface-active compounds that, apart from a C₈₋₂₄ alkyl or acyl group, have at least one free amino group and at least one —COOH or —SO₃H group in the molecule, and are able to form internal salts. Examples of suitable ampholytes are N-alkylglycines, N-alkyl propionic acids, N-alkylamino butyric acids, N-alkylimino dipropionic acids, N-hydroxyethyl-N-alkylamidopropylglycines, N-alkyltaurines, N-alkylsarcosines, 2-alkylamino propionic acids and alkylamino acetic acids, each with about 8 to 24 carbon atoms in the alkyl group. Particularly preferred ampholytes are N-cocoalkylamino propionate, cocoacylaminoethylamino propionate and C₁₂-C₁₈ acyl sarcosine.

Agents according to the invention contain the ingredients or active substances in a cosmetically acceptable carrier.

Preferred cosmetically acceptable carriers are aqueous, alcoholic or aqueous alcoholic media containing preferably at least 10 wt. % water, based on total composition. In particular, lower alcohols containing 1 to 4 carbon atoms, such as ethanol and isopropanol, typically used for cosmetic purposes, can be used as alcohols. It is inventively preferred to incorporate at least one (C₁ to C₄) monoalkyl alcohol in the agents, particularly in an amount of 1 to 50 wt. %, especially 5 to 30 wt. %. Again, this is particularly preferred when manufacturing pump foams or aerosol foams.

Organic solvents or mixture of solvents with a boiling point of less than 400° C. can be used as additional co-solvents in an amount of 0.1 to 15 wt. %, preferably 1 to 10 wt. %, based on total weight of the agent. Particularly suitable additional co-solvents are unbranched or branched hydrocarbons such as pentane, hexane, isopentane and cyclic hydrocarbons such as cyclopentane and cyclohexane. Additional, particularly preferred water-soluble solvents are glycerin, ethylene glycol and propylene glycol in an amount of up to 30 wt. %, based on total agent.

In particular, the addition of glycerin and/or propylene glycol and/or polyethylene glycol and/or polypropylene glycol increases the flexibility of the polymer film formed when the agent is used. Consequently, if a more flexible hold is desired, then the agents preferably contain 0.01 to 30 wt. % glycerin and/or propylene glycol and/or polyethylene glycol and/or polypropylene glycol, based on total weight of the agent.

The agents preferably have a pH of 2 to 11. The pH range is particularly preferably from 2 to 8. In the context of this publication, pH data refers to the pH at 25° C. unless otherwise stated.

Agents according to the invention can additionally contain auxiliaries and additives that are typically incorporated into conventional styling agents.

In particular, care products can be mentioned as suitable auxiliaries and additives.

Silicone oil and/or silicone gum, for example, can be employed as the care substance.

Suitable silicone oils or gums include dialkyl and alkylarylsiloxanes, such as dimethylpolysiloxane and methylphenylpolysiloxane, as well as their alkoxylated, quatemized or also anionic derivatives. Cyclic and linear polydialkylsiloxanes, their alkoxylated and/or aminated derivatives, dihydroxypolydimethylsiloxanes and polyphenylalkylsiloxanes are preferred.

Silicone oils afford a wide variety of effects. Thus, for example, they simultaneously influence dry and wet combability, feel of the dry and wet hair, as well as gloss. The term “silicone oils” is understood by one skilled in the art to mean organosilicon compounds with a plurality of structures. In the first instance they include Dimethiconols. The following commercial products are given as examples of such products: Botanisil NU-150M (Botanigenics), Dow Corning 1-1254 Fluid, Dow Corning 2-9023 Fluid, Dow Corning 2-9026 Fluid, Ultrapure Dimethiconol (Ultra Chemical), Unisil SF-R (Universal Preserve), X-21-5619 (Shin-Etsu Chemical Co.), Abil OSW 5 (Degussa Care Specialties), ACC DL-9430 Emulsion (Taylor Chemical Company), AEC Dimethiconol & Sodium Dodecylbenzene sulfonate (A & E Connock (Perfumery & Cosmetics) Ltd.), B C Dimethiconol Emulsion 95 (Basildon Chemical Company, Ltd.), Cosmetic Fluid 1401, Cosmetic Fluid 1403, Cosmetic Fluid 1501, Cosmetic Fluid 1401 DC (all from Chemsil Silicones, Inc.), Dow Corning 1401 Fluid, Dow Corning 1403 Fluid, Dow Corning 1501 Fluid, Dow Corning 1784 HVF Emulsion, Dow Corning 9546 Silicone Elastomer Blend (all from Dow Corning Corporation), Dub Gel S1 1400 (Stearinerie Dubois FiIs), HVM 4852 Emulsion (Crompton Corporation), Jeesilc 6056 (Jeen International Corporation), Lubrasil, Lubrasil DS (both from Guardian Laboratories), Nonychosine E, Nonychosine V (both from Exsymol), SanSurf Petrolatum-25, Satin Finish (both from Collaborative Laboratories, Inc.), Silatex-D30 (Cosmetic Ingredient Resources), Silsoft 148, Silsoft E-50, Silsoft E-623 (all from Crompton Corporation), SM555, SM2725, SM2765, SM2785 (all from GE Silicones), Taylor T-SiI CD-1, Taylor TME-4050E (all from Taylor Chemical Company), TH V 148 (Crompton Corporation), Tixogel CYD-1429 (Sud-Chemie Performance Additives), Wacker-Belsil CM 1000, Wacker-Belsil CM 3092, Wacker-Belsil CM 5040, Wacker-Belsil DM 3096, Wacker-Belsil DM 3112 VP, Wacker-Belsil DM 8005 VP, Wacker-Belsil DM 60081 VP (all from Wacker-Chemie GmbH).

Dimethicones form the second group of silicones that can be used according to the invention. They can be linear, branched, cyclic, or cyclic and branched.

Dimethicone copolyols (S3) form a further group of suitable silicones. Suitable Dimethicone copolyols are commercially available and are marketed, for example, by Dow Corning under the trade name Dow Coming® 5330 Fluid.

Naturally, the Dimethiconols, Dimethicones and/or Dimethicone copolyols can already be present as an emulsion. The corresponding emulsions of Dimethiconols, Dimethicones and/or Dimethicone copolyols can be produced both after production of the corresponding Dimethiconols, Dimethicones and/or Dimethicone copolyols from these and typical emulsification processes known to one skilled in the art. Cationic, anionic, non-ionic or zwitterionic surfactants and emulsifiers can be used as auxiliaries and adjuvants for the production of the corresponding emulsions. Naturally, emulsions of Dimethiconols, Dimethicones and/or Dimethicone copolyols can also be produced by an emulsion polymerization process. These types of processes are also well known to one skilled in the art.

When Dimethiconols, Dimethicones and/or Dimethicone copolyols are used as an emulsion, then the droplet size of the emulsified particles ranges from 0.01 to 10,000 μm, preferably 0.01 to 100 μm, particularly preferably 0.01 to 20 μm and quite particularly preferably 0.01 to 10 μm. Particle size is determined here according to the light scattering method.

If branched Dimethiconols, Dimethicones and/or Dimethicone copolyols are used, then it can be taken as understood that the branching is greater than a fortuitous branching that accidentally results from impurities in the respective monomers. Accordingly, the degree of branching is understood to be greater than 0.01% for branched Dimethiconols, Dimethicones and/or Dimethicone copolyols. The degree of branching is preferably greater than 0.1% and quite particularly preferably greater than 0.5%. The degree of branching is determined from the ratio of the unbranched monomers to the branched monomers (i.e., the amount of tri- and tetrafunctional siloxanes). Accordingly, both low-branched as well as highly branched Dimethiconols, Dimethicones and/or Dimethicone copolyols can be quite particularly preferred.

Further suitable silicones are amino-functional silicones, especially the silicones compiled under the INCI name Amodimethicone. Consequently, it is inventively preferred when the agents according to the invention additionally comprise at least one amino-functional silicone. These are understood to be silicones that have at least one, optionally substituted, amino group. These silicones are designated as Amodimethicones according to INCI nomenclature and are available, for example, in the form of an emulsion as the commercial product Dow Corning® 939 or as the commercial product Dow Corning® 949 in a mixture with a cationic and a non-ionic surfactant.

Preferably, those amino functional silicones are used which have an amine number of 0.25 meq/g or greater, preferably 0.3 meq/g or greater, and particularly preferably 0.4 meq/g or greater. The amine number is the milli-equivalents of amine per gram of the amino functional silicone. It can be measured by titration and can also be reported with the unit mg KOH/g.

The agents preferably contain silicones in amounts of 0.01 wt. % to 15 wt. %, particularly preferably in amounts of 0.05 to 2 wt. %, based on total weight of the agent.

The composition can include at least one protein hydrolyzate and/or one of its derivatives as a care substance of another compound class.

Protein hydrolyzates are product mixtures obtained by acid-, base- or enzyme-catalyzed degradation of proteins (albumins). According to the invention, the term “protein hydrolyzates” refers to total hydrolyzates as well as individual amino acids and their derivatives as well as mixtures of different amino acids. The molecular weight of useful protein hydrolyzates ranges from 75, the molecular weight of glycine, to 200,000, preferably 75 to 50,000, and more preferably 75 to 20,000 Dalton.

According to the invention, the added protein hydrolyzates can be of vegetal, as well as animal or marine or synthetic origin.

Animal protein hydrolyzates include elastin, collagen, keratin, silk protein, and milk albumin protein hydrolyzates, which can also be present in the form of their salts. Such products are marketed, for example, under the trade names Dehylan® (Cognis), Promois® (Interorgana), Collapuron® (Cognis), Nutrilan® (Cognis), Gelita-Sol® (Deutsche Gelatine Fabriken Stoess & Co), Lexein® (Inolex), Sericin (Pentapharm) and Kerasol® (Croda).

The agents contain protein hydrolyzates, for example, in concentrations of 0.01 wt. % to 20 wt. %, preferably 0.05 wt. % up to 15 wt. % and more preferably in amounts of 0.05 wt. % up to 5 wt. %, based on total end-use preparation.

Agents according to the invention can further contain at least one vitamin, one provitamin, one vitamin precursor and/or one of their derivatives as the care substance.

Preferred vitamins, provitamins and vitamin precursors are those normally classified in the groups A, B, C, E, F and H.

Retinol (vitamin A₁) as well as 3,4-didehydroretinol, (vitamin A₂) belong in the group of substances designated as vitamin A. β-carotene is the provitamin of retinol. Examples of suitable vitamin A components are vitamin A acid and its esters, vitamin A aldehyde and vitamin A alcohol as well as its esters such as the palmitate and acetate. The agents preferably contain vitamin A components in amounts of 0.05 to 1 wt. %, based on total application preparation.

The vitamin B group or the vitamin B complex include inter alia vitamin B₁ (thiamine), vitamin B₂ (riboflavin), vitamin B₃ (nicotinic acid and/or nicotinic acid amide (niacinamide)), vitamin B₅ (pantothenic acid, panthenol and pantolactone), vitamin B₆ (pyridoxine as well as pyridoxamine and pyridoxal), vitamin C (ascorbic acid), vitamin E (tocopherols, especially a-tocopherol), vitamin F (linoleic acid and/or linolenic acid), vitamin H.

The agents preferably contain vitamins, provitamins and vitamin precursors from groups A, B, C, E and H. Panthenol, pantolactone, pyridoxine and its derivatives as well as nicotinamide and biotin are especially preferred.

D-panthenol is quite particularly preferably used as a care substance, optionally in combination with at least one of the abovementioned silicone derivatives.

Like the addition of glycerin and/or propylene glycol, the addition of panthenol increases the flexibility of the polymer film formed when the agent is used. Thus, if a particularly flexible hold is desired, then the agents can contain panthenol instead of or in addition to glycerin and/or propylene glycol. In a preferred embodiment, the agents contain panthenol, preferably in a quantity of 0.05 to 10 wt. %, particularly preferably 0.1 to 5 wt. %, based on total agent.

Agents according to the invention can further contain at least one plant extract as a care substance.

Usually, these extracts are manufactured by extraction of the whole plant. In individual cases, however, it can be preferred to produce the extracts solely from blossoms and/or leaves of the plant.

According to the invention, extracts mainly from green tea, oak bark, stinging nettle, hamamelis, hops, henna, chamomile, burdock root, field horsetail, hawthorn, linden flowers, almonds, aloe vera, spruce needles, horse chestnut, sandal wood, juniper, coconut, mango, apricot, lime, wheat, kiwi, melon, orange, grapefruit, sage, rosemary, birch, malva, lady's smock, common yarrow, thyme, lemon balm, rest-harrow, coltsfoot, marshmallow (althea), meristem, ginseng and ginger are preferred.

In addition, it can be preferred to use mixtures of a plurality, particularly two different plant extracts in the agents.

Mono- or oligosaccharides can also be used as a care substance in agents according to the invention.

Both monosaccharides and oligosaccharides, such as raw sugar, lactose and raffinose can be used.

According to the invention, use of monosaccharides is preferred. Once again, the monosaccharides preferably include those compounds having 5 or 6 carbon atoms.

Suitable pentoses and hexoses are ribose, arabinose, xylose, lyxose, allose, altrose, glucose, mannose, gulose, idose, galactose, talose, fucose and fructose. Arabinose, glucose, galactose and fructose are preferred carbohydrates; glucose is particularly preferably used, and is suitable both in the D-(+) or L-(−) configuration or as the racemate.

In addition, derivatives of these pentoses and hexoses can also be used, such as the corresponding onic and uronic acids (sugar acids), sugar alcohols, and glycosides.

Preferred sugar acids are gluconic acid, glucuronic acid, sugar acids, mannosugar acids and mucic acids. Preferred sugar alcohols are sorbitol, mannitol and dulcitol.

Preferred glycosides are methyl glucosides.

As incorporated mono- and oligosaccharides are usually obtained from natural raw materials such as starch, they generally possess configurations corresponding to these raw materials (e.g., D-glucose, D-fructose and D-galactose).

The agents preferably contains mono- or oligosaccharides in an amount of 0.1 to 8 wt. %, particularly preferably 1 to 5 wt. %, based on total end-use preparation.

The agent can further include at least one lipid as a care substance.

According to the invention, suitable lipids are phospholipids, for example, soy lecithin, egg lecithin, and cephalins, as well as substances known under the INCI names Linoleamidopropyl PG-Dimonium Chloride Phosphate, Cocamidopropyl PG-Dimonium Chloride Phosphate and Stearamidopropyl PG-Dimonium Chloride Phosphate. These are commercialized, for example, by the Mona Company under the trade names Phospholipid EPA®, Phospholipid PTC® and Phospholipid SV®. The agents preferably contain lipids in amounts of 0.01 to 10 wt. %, particularly 0.1 to 5 wt. %, based on total end-use preparation.

Oil bodies are also suitable as a care substance.

Natural and synthetic cosmetic oil bodies include:
vegetal oils. Examples of such oils are sunflower oil, olive oil, soya oil, rapeseed oil, almond oil, jojoba oil, orange oil, wheat germ oil, peach stone oil and the liquid parts of coconut oil. Other triglyceride oils such as the liquid fractions of beef tallow as well as synthetic triglyceride oils are also suitable.
liquid paraffin oils, isoparaffin oils and synthetic hydrocarbons as well as di-n-alkyl ethers containing 12 to 36 carbon atoms, particularly 12 to 24 carbon atoms such as di-n-octyl ether, di-n-decyl ether, di-n-nonyl ether, di-n-undecyl ether, di-n-dodecyl ether, n-hexyl n-octyl ether, n-octyl n-decyl ether, n-decyl n-undecyl ether, n-undecyl n-dodecyl ether and n-hexyl n-undecyl ether and di-tert.butyl ether, diisopentyl ether, di-3-ethyldecyl ether, tert.butyl n-octyl ether, isopentyl n-octyl ether and 2-methylpentyl n-octyl ether. The commercial products of the compounds 1,3-di-(2-ethylhexyl)cyclohexane (Cetiol® S) and di-n-octyl ether (Cetiol® OE) can be preferred.
Ester oils. Ester oils refer to the esters of C₆-C₃₀ fatty acids with C₂-C₃₀ fatty alcohols. The monoesters of fatty acids with alcohols containing 2 to 24 carbon atoms are preferred. According to the invention, isopropyl myristate (Rilanie IPM), isononanoic acid C16-18 alkyl ester (Cetiol® SN), 2-ethylhexyl palmitate (Cegesoft® 24), stearic acid 2-ethylhexyl ester (Cetiol 868), cetyl oleate, glycerine tricaprylate, cocofatty alcohol caprinate/-caprylate (Cetiol® LC), n-butyl stearate, oleyl erucate (Cetiol® J 600), isopropyl palmitate (Rilanit® IPP), oleyl oleate (Cetiol®), lauric acid hexyl ester (Cetiol® A), di-n-butyl adipate (Cetiol® B), myristyl myristate (Cetiol® MM), cetearyl isononanoate (Cetiol® SN), oleic acid decyl ester (Cetiol® V) are particularly preferred.
Dicarboxylic acid esters such as di-n-butyl adipate, di-(2-ethylhexyl)adipate, di-(2-ethylhexyl)succinate and di-isotridecyl acetate as well as diol esters such as ethylene glycol dioleate, ethylene glycol di-isotridecanoate, propylene glycol di(2-ethylhexanoate), propylene glycol di-isostearate, propylene glycol di-pelargonate, butane diol di-isostearate, neopentyl glycol dicaprylate,
symmetrical, unsymmetrical or cyclic esters of carbon dioxide with fatty alcohols, for example, as described in DE-OS 197 56 454, glycerine carbonate or dicaprylyl carbonate (Cetiol® CC),
trifatty acid esters of saturated and/or unsaturated linear and/or branched fatty acids with glycerin,
fatty acid partial glycerides, under which are understood monoglycerides, diglycerides and their industrial mixtures. When using industrial products, minor amounts of triglycerides may still be contained as a result of the production process. The partial glycerides preferably comply with the Formula (D4-I),

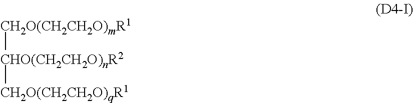

wherein R¹, R² and R³ are, independently of each other, hydrogen or a linear or branched, saturated and/or unsaturated acyl group with 6 to 22 carbon atoms, preferably 12 to 18 carbon atoms, with the proviso that at least one of these groups is an acyl group and at least one of these groups is hydrogen. The sum of (m+n+q) is 0 or a number from 1 to 100, preferably 0 or 5 to 25. Preferably, R¹ is an acyl group, R² and R³ are hydrogen, and the sum of (m+n+q) is 0. Typical examples are mono- and/or diglycerides based on caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselic acid, linoleic acid, linolenic acid, elaeostearic acid, arachidonic acid, gadoleic acid, behenic acid and erucic acid as well as their industrial mixtures. Oleic acid monoglycerides are preferably employed.

The added amount of natural and synthetic cosmetic oil bodies in the agents is usually 0.1 to 30 wt. %, based on total end-use preparation, preferably 0.1 to 20 wt. % and particularly 0.1 to 15 wt. %.

Although each of the cited care substances alone already provides a satisfactory result, according to the present invention all embodiments are included wherein the agent has a plurality of conditioners, even from different groups.

By addition of a UV filter, both the agent as well as the treated fibers can be protected against damage from UV radiation. Consequently, at least one UV filter is preferably added to the agent. Suitable UV filters are not generally limited with respect to structure and physical properties. Indeed, all UV filters that can be used in the cosmetic field having an absorption maximum in the UVA (315-400 nm), UVB (280-315 nm) or UVC (<280 nm) regions are suitable. UV filters having an absorption maximum in the UVB region, especially in the range from about 280 to about 300 nm, are particularly preferred.

Preferred UV-filters are chosen from substituted benzophenones, p-aminobenzoates, diphenylacrylates, cinnamates, salicylates, benzimidazoles and o-aminobenzoates.

Exemplary usable UV-filters are 4-amino-benzoic acid, N,N,N-trimethyl-4-(2-oxoborn-3-ylidenemethyl)aniline methyl sulfate, 3,3,5-trimethylcyclohexyl salicylate (Homosalate), 2-hydroxy-4-methoxy-benzophenone (Benzophenone-3; Uvinul® M 40, Uvasorb® MET, Neo Heliopan® BB, Eusolex® 4360), 2-phenylbenzimidazol-5-sulfonic acid and their potassium, sodium and triethanolamine salts (phenylbenzimidazole sulfonic acid; Parsol® HS; Neo Heliopan® Hydro), 3,3′-(1,4-phenylenedimethylene)-bis(7,7-dimethyl-2-oxo-bicyclo-[2.2.1]hept-1-yl-methanesulfonic acid) and their salts, 1-(4-tert.-butylphenyl)-3-(4-methoxyphenyl)propane-1,3-dione(butylmethoxydibenzoylmethane; Parsol® 1789, Eusolex® 9020), a-(2-oxoborn-3-ylidene)-toluene-4-sulfonic acid and salts thereof, ethoxylated ethyl 4-aminobenzoate (PEG-25 PABA; Uvinul® P 25), 2-ethylhexyl 4-dimethylaminobenzoate (Octyl Dimethyl PABA; Uvasorb® DMO Escalol® 507, Eusolex® 6007), 2-ethylhexyl salicylate (Octyl Salicylate; Escalol® 587, Neo Heliopan® OS, Uvinul® 018), isopentyl 4-methoxycinnamate(isoamyl p-methoxycinnamate; Neo Heliopan® E 1000), 2-ethylhexyl 4-methoxycinnamate (Octyl Methoxycinnamate; Parsol® MCX, Escalol® 557, Neo Heliopan® AV), 2-hydroxy-4-methoxybenzophenone-5-sulfonic acid and sodium salts thereof, (benzophenone-4; Uvinul® MS 40; Uvasorb® S 5), 3-(4′-methylbenzylidene)-D,L-camphor (4-methylbenzylidene camphor; Parsol® 5000, Eusolex® 6300), 3-benzylidene-camphor (3-Benzylidene camphor), 4-isopropylbenzyl salicylate, 2,4,6-trianilino-(p-carbo-2′-ethylhexyl-1′-oxy)-1,3,5-triazine, 3-imidazol-4-yl-acrylic acid and its ethyl ester, polymers of N-{(2 and 4)-[2-oxoborn-3-ylidenemethyl]benzyl}-acrylamide, 2,4-dihydroxybenzophenone (Benzophenone-1; Uvasorb® 20 H, Uvinul® 400), 2-ethylhexyl ester of 1,1′-diphenylacrylonitrilic acid (Octocrylene; Eusolex® OCR, Neo Heliopan® Type 303, Uvinul® N 539 SG), menthyl o-aminobenzoate (menthyl anthranilate; Neo Heliopan® MA), 2,2′,4,4′-tetrahydroxybenzophenone (Benzophenone-2, Uvinul® D-50), 2,2′-dihydroxy-4,4′-dimethoxybenzophenone (Benzophenone-6), sodium 2,2′-dihydroxy-4,4′-dimethoxybenzophenone-5-sulfonate and 2′-ethylhexyl 2-cyano-3,3-diphenylacrylate. 4-Amino-benzoic acid, N,N,N-trimethyl-4-(2-oxobom-3-ylidenemethyl)aniline methyl sulfate, 3,3,5-trimethyl-cyclohexyl salicylate, 2-hydroxy-4-methoxy-benzophenone, 2-phenylbenzimidazole-5-sulfonic acid and the potassium, sodium and triethanolamine salts thereof, 3,3′-(1,4-phenylenedimethylene)-bis(7,7-dimethyl-2-oxo-bicyclo-[2.2.1]hept-1-yl methane sulfonic acid) and salts thereof, 1-(4-tert.-butylphenyl)-3-(4-methoxyphenyl)-propane-1,3-dione, a-(2-oxoborn-3-ylidene)-toluene-4-sulfonic acid and salts thereof, ethoxylated ethyl 4-aminobenzoate, 2-ethylhexyl 4-dimethylaminobenzoate, 2-ethylhexyl salicylate, isopentyl 4-methoxycinnamate, 2-ethylhexyl 4-methoxycinnamate, 2-hydroxy-4-methoxybenzophenone-5-sulfonic acid and its sodium salt, 3-(4′-methylbenzylidene)-D,L-camphor, 3-benzylidene-camphor, 4-isopropylbenzyl salicylate, 2,4,6-tri-anilino-(p-carbo-2′-ethylhexyl-1′-oxy)-1,3,5-triazine, 3-imidazol-4-yl-acrylic acid and its ethyl ester, polymers of N-{(2 and 4)-[2-oxoborn-3-ylidenemethyl]benzyl}acrylamide are preferred. According to the invention, 2-hydroxy-4-methoxy-benzophenone, 2-phenylbenzimidazole-5-sulfonic acid and its potassium, sodium and triethanolamine salts, 1-(4-tert.-Butylphenyl)-3-(4-methoxyphenyl)-propane-1,3-dione, 4-methoxycinnamic acid 2-ethylhexyl ester and 3-(4′-methylbenzylidene)-D,L-camphor are quite particularly preferred.

The agent usually contain UV filters in amounts of 0.01 to 5 wt. %, based on total end-use preparation. Quantities of 0.1-2.5 wt. % are preferred.

In a particular embodiment, the agent further includes one or more substantive dyes. Application of the agent then enables the treated keratinic fiber not only to be temporarily styled but also to be dyed at the same time. This can be particularly desirable when only a temporary dyeing is desired, for example, with flamboyant fashion colors that can be subsequently removed from the keratinic fibers by simply washing them out.

Substantive dyes are usually nitrophenylenediamines, nitroamino phenols, azo dyes, anthraquinones or indophenols. Preferred substantive dyestuffs are compounds with the international designations or trade names HC Yellow 2, HC Yellow 4, HC Yellow 5, HC Yellow 6, HC Yellow 12, Acid Yellow 1, Acid Yellow 10, Acid Yellow 23, Acid Yellow 36, HC Orange 1, Disperse Orange 3, Acid Orange 7, HC Red 1, HC Red 3, HC Red 10, HC Red 11, HC Red 13, Acid Red 33, Acid Red 52, HC Red 13N, Pigment Red 57:1, HC Blue 2, HC Blue 11, Disperse Blue 12, Acid Blue 3, Acid Green 7, Acid Green 50, HC Violet 1, Disperse Violet 1, Disperse Violet 4, Acid Violet 43, Disperse Black 9, Acid Black 1, and Acid Black 52 known compounds as well as 1,4-diamino2-nitrobenzene, 2-amino-4-nitrophenol, 1,4-bis-(β-hydroxyethyl)-amino-2-nitrobenzene, 3-nitro-4-(β-hydroxyethyl)-amino phenol, 2-(2′-hydroxyethypamino-4,6-dinitrophenol, 1-(2′-hydroxyethyl)amino-4-methyl-2-nitrobenzene, 1-amino-4-(2′-hydroxyethyl)-amino-5-chloro-2-nitrobenzene, 4-amino-3-nitrophenol, 1-(2′-ureidoethyl)amino-4-nitrobenzene, 4-amino-2-nitrodiphenylamine-2′-carboxylic acid, 6-nitro-1,2,3,4-tetrahydroquinoxaline, 2-hydroxy-1,4-naphthoquinone, picramic acid and its salts, 2-amino-6-chloro-4-nitrophenol, 4-ethylamino-3-nitrobenzoic acid and 2-chloro-6-ethylamino-1-hydroxy-4-nitrobenzene. Cationic substantive dyes are preferably employed. Particular preference is given here to
(a) cationic triphenylmethane dyes, such as Basic Blue 7, Basic Blue 26, Basic Violet 2 and Basic Violet 14,
(b) aromatic systems which are substituted by a quaternary nitrogen group, such as Basic Yellow 57, Basic Red 76, Basic Blue 99, Basic Brown 16 and Basic Brown 17, and
(c) substantive dyes having a heterocycle that has at least one quaternary nitrogen atom, as specified, for example, in EP-A2-998 908 in claims 6 to 11.

Dyes also known under the names Basic Yellow 87, Basic Orange 31 and Basic Red 51, are quite particularly preferred cationic substantive dyes of group (c). Cationic substantive dyes commercialized under the trade name Arianor® are likewise quite particularly preferred cationic substantive dyes.

Agents according to this embodiment contain substantive dyes preferably in an amount of 0.001 to 20 wt. %, based on total agent.

Preferably, the agents are exempt from oxidation dye precursors. Oxidation dye precursors are divided into developer components and coupler components. Under the influence of oxidizing agents or from atmospheric oxygen, the developer components form the actual colorants among each other or by coupling with one or more coupler components.

Formulation of the agents can be in all usual forms for styling agents, for example, in the form of solutions applied as hair water or pump or aerosol spray onto the hair, in the form of creams, emulsions, waxes, gels, or also surfactant-containing foaming solutions or other preparations suitable for application on the hair.

Hair creams and hair gels generally contain structurants and/or thickening polymers which give the desired consistency to the products. Structurants and/or thickening polymers are typically added in amounts of 0.1 to 10 wt. %, based on total product. Quantities of 0.5 to 5 wt. %, particularly 0.5 to 3 wt. %, are preferred.

The agents are preferably a pump spray, aerosol spray, pump foam or aerosol foam.

For this the agents are packed in a dispensing device, illustrated by either a pressurized gas container additionally containing a propellant (“aerosol container”) or by a non-aerosol container.

Pressurized gas containers, by which a product is dispersed through a valve by the internal gas pressure in the container, are defined as “aerosol containers”. The opposite of the aerosol definition, a container under normal pressure, is defined as a “non-aerosol container”, from which a product is dispersed by mechanical actuation of a pump system.

The agents are particularly preferably packed as an aerosol hair foam or aerosol hair spray. Consequently, the agent additionally has at least one propellant.

Inventively suitable exemplary propellants are chosen from N₂O, dimethyl ether, CO₂, air, alkanes containing 3 to 5 carbon atoms, such as propane, n-butane, iso-butane, n-pentane and iso-pentane, and their mixtures. Dimethyl ether, propane, n-butane, iso-butane and their mixtures are preferred.

According to a preferred embodiment, the cited alkanes, mixtures of the cited alkanes or mixtures of the cited alkanes with dimethyl ether are preferred as the sole propellant. However, the invention also includes joint utilization with propellants of the fluorochlorohydrocarbon type, especially fluorinated hydrocarbons.

Regarding the weight ratio of propellant to the usual ingredients of the preparation, the size of the aerosol droplets or foam bubbles and the relevant size distribution can be adjusted for a given spray device.

The amount of added propellant varies as a function of the actual composition of the agent, packaging used and the desired product type, for example, hair spray or hair foam. When a conventional spray device is used, aerosol foam products preferably contain propellant in amounts of 1 to 35 wt. %, based on total product. Quantities of 2 to 30 wt. %, especially 3 to 15 wt. %, are particularly preferred. Aerosol sprays generally contain greater amounts of propellant. Here, the propellant is preferably added in amounts of 30 to 98 wt. %, based on total product. Quantities of 40 to 95 wt. %, especially 50 to 95 wt. %, are particularly preferred.

The aerosol products can be manufactured according to conventional techniques. Generally, all ingredients of the agent except the propellant are charged into a suitable pressure-resistant container. This is then sealed with a valve. The desired amount of propellant is then filled by conventional techniques. Agents in the form of gels are foamed in a two-chamber aerosol container, preferably with isopentane as the propellant, which is added to the agent and packed in the first chamber of the two-chamber aerosol container. At least one additional propellant different from isopentane is packed in the second chamber of the two-chamber aerosol container and generates a higher pressure than the isopentane. Propellants of the second chamber are preferably chosen from N₂O, dimethyl ether, CO₂, air, alkanes containing 3 or 4 carbon atoms (such as propane, n-butane, iso-butane) as well as mixtures thereof.

Aerosol hair foams or aerosol hair sprays containing the above described agent and at least one propellant are a preferred embodiment of the agent according to the invention.

Preferred agents and propellants of the aerosol hair foam or aerosol hair spray, as well as the relevant amounts of propellant correspond to those already mentioned above.

A second subject matter of the invention is the use of the agent according to the invention for the temporary shaping of hair and/or for hair care.

The agents and products containing these agents, especially aerosol hair foams or aerosol hair sprays, give treated hair a very strong, long-lasting hold to the hairstyle, while the hair remains flexible. If the agent is made up as hair foam, then a stable, micro-porous and creamy foam is formed that can be uniformly dispersed on the hair without dripping.

A third subject matter of the invention is a method for treating keratin-containing fibers, especially human hair, wherein an agent according to the first subject matter is foamed to a foam by a dispensing device, and the resulting foam applied onto the keratin-containing fibers.

It is inventively preferred here that the keratin-containing fibers are shaped and this shape is fixed by the agent of the first subject matter of the invention.

The abovementioned dispensing devices (see above) are inventively preferred.

A fourth subject matter of the invention is a method for treating keratin-containing fibers, especially human hair, wherein an agent according to the first subject matter is applied as a spray onto the keratin-containing fibers by a dispensing device.

It is inventively preferred here that the keratin-containing fibers are shaped and this shape is fixed by the agent of the first subject matter of the invention.

The abovementioned dispensing devices (see above) are inventively preferred.

The following examples are intended to illustrate the subject matter of the present invention in more detail, without limiting it in any way.

### EXAMPLES

Unless otherwise stated, the quantities are understood to be in weight per cent.

The following formulations were prepared by blending the listed raw materials:

| Raw materials | A | B | C | D | E |
|---|---|---|---|---|---|
| Hydagen ® HCMF | 0.2 | 0.5 | 0.7 | 0.3 | 0.5 |
| Lactic acid | 0.1 | 0.2 | 0.4 | 0.1 | 0.25 |
| Aquastyle ® 300 | 3.0 | 2.0 | 1.0 | 4.0 | 3.5 |
| Luviskol ® 64 W | — | 5.0 | — | — | — |
| Aquaflex ® XL-30 ¹ | — | — | 3 | — | — |
| Celquat ® L-200 | — | — | — | 0.5 | — |
| PEG-40 hydrogenated castor oil | 0.1 | 0.2 | 0.2 | 0.2 | 0.1 |

| | |
|---|---|
| Water | ←-------------ad 100 -----------→ |

¹ Copolymer of 2,5-furan dione with 2-methyl-1-propene, N,N-dimethyl-1,3- propylenediamine, polyethylene-polypropylene glycol 2-aminopropyl methyl ether (Chemical Abstracts Number: 497926-97-3) (28 to 32 wt. % active substance, INCI name: Polyimide-1) (ISP)

The formulations A to E were each filled into an aerosol container that meets the following technical parameter: aluminum reservoir container with valve product 522983 PV 10697 from the Precision Company (Deutsche Präzisions-Ventil GmbH).

The aerosol container was filled with a mixture of the propellant gases propane/butane (47 wt. % propane, 50 wt. % butane, 3 wt. % isobutene), such that the weight ratio of the formulation to the propellant gas was 92 to 8.

All formulations when applied onto the hair produced an outstandingly flexible hold to the hairstyle. The hair received a very good care.

All formulations were free of turbidity.

We claim:

1. Agent for treating keratin-containing fibers comprising in a cosmetically acceptable carrier:
(a) at least one amphiphilic, cationic polymer having at least one structural unit of Formula (I), at least one structural unit of Formula (II), at least one structural unit of Formula (III) and at least one structural unit of Formula (IV), 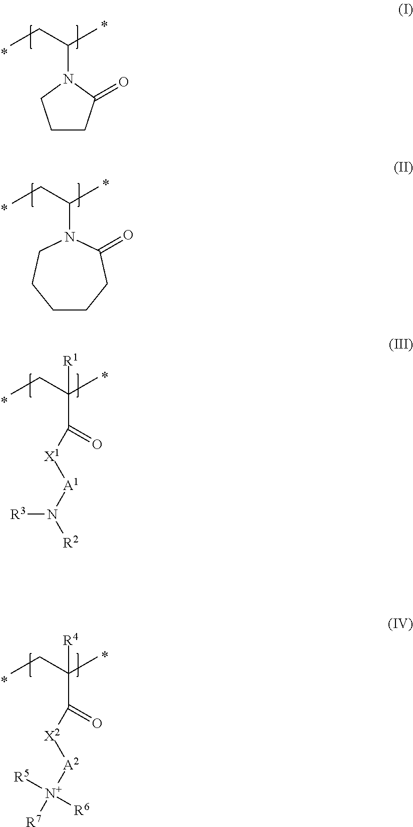
wherein
R¹ and R⁴ are, independently of one another, a hydrogen atom or a methyl group,
X¹ and X² are, independently of one another, an oxygen atom or an NH group,
A¹ and A² are, independently of one another, an ethane-1,2-diyl, propane-1,3-diyl or butane-1,4-diyl group,
R², R³, R⁵ and R⁶ are, independently of one another, a (C₁ to C₄) alkyl group, and
R⁷ is a (C₈ to C₃₀) alkyl group, and
(b) at least one film-forming and/or setting polymer chosen from chitosan and its derivatives.

2. Agent according to claim 1 wherein R¹ and R⁴ are each a methyl group.

3. Agent according to claim 1 wherein A¹ and A² are, independently of one another, ethane-1,2-diyl or propane-1,3-diyl.

4. Agent according to claim 1 wherein X¹ is an NH group.

5. Agent according to claim 1 wherein X² is an NH group.

6. Agent according to claim 1 wherein R², R³, R⁵ and R⁶ are, independently of one another, methyl or ethyl.

7. Agent according to claim 1 wherein R⁷ is a (C₁₀ to C₂₄) alkyl group.

8. Agent according to claim 1 wherein R⁷ is decyl(caprinyl), dodecyl(lauryl), tetradecyl(myristyl), hexadecyl(cetyl), octadecyl(stearyl), eicosyl(arachyl) or docosyl(behenyl).

9. Agent according to claim 1 wherein the at least one amphiphilic, cationic polymer comprises at least one structural unit of Formula (I), at least one structural unit of Formula (II), at least one structural unit of Formula (III-8), and at least one structural unit of Formula (IV-8) 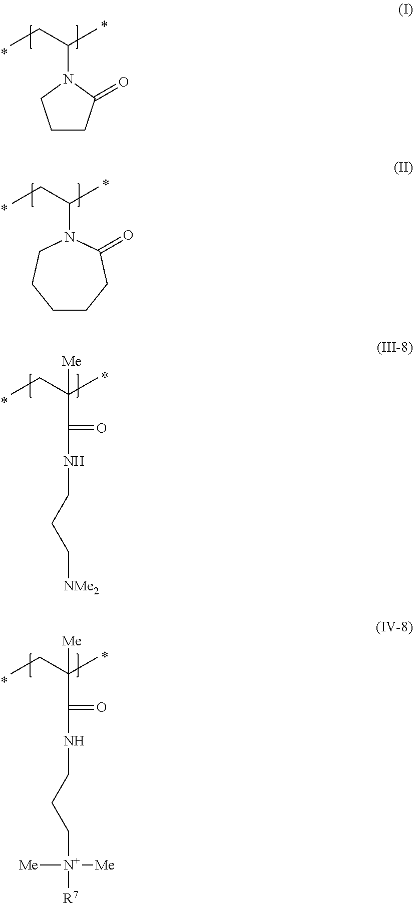
wherein R⁷ is octyl(capryl), decyl(caprinyl), dodecyl(lauryl), tetradecyl(myristyl), hexadecyl(cetyl), octadecyl(stearyl), eicosyl(arachyl) or docosyl(behenyl).

10. Agent according to claim 1 wherein the at least one amphiphilic, cationic polymer is present in an amount of 0.1 wt. % to 20.0 wt. %, based on total weight of the agent.

11. Agent according to claim 1 further comprising neutralization products of chitosan with at least one acid as the chitosan derivative(s).

12. Agent according to claim 1 wherein the film-forming and/or setting polymers chosen from chitosan and its derivatives are present in an amount of 0.01 wt. % to 20 wt. %, based on total weight of the agent.

13. Agent according to claim 1 further comprising at least one surfactant.

14. Aerosol foam or spray comprising the agent according to claim 1.

15. Method for treating keratin-containing fibers comprising foaming an agent according to claim 1 to a foam using a dispensing device, and applying the resulting foam onto the keratin-containing fibers.

16. Method for treating keratin-containing fibers comprising applying an agent according to claim 1 as a spray onto the keratin-containing fibers using a dispensing device.
